(19) United States
(12) Patent Application Publication
(10) Pub. No.: US 2003/0082156 A1
(43) Pub. Date: May 1, 2003

(54) Polypeptides and methods for thymic vaccination

(75) Inventors: Ellis L. Reinherz, Lincoln, MA (US); Tetsuro Sasada, Nakagyo-ku (JP); Jia-huai Wang, Belmont, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Dana-Farber Cancer Institute, Inc., Boston, MA

(21) Appl. No.: 10/155,879

(22) Filed: May 23, 2002

Related U.S. Application Data
(63) Continuation of application No. PCT/US00/31502, filed on Nov. 16, 2000.
(60) Provisional application No. 60/168,167, filed on Nov. 30, 1999.
(60) Provisional application No. 60/167,378, filed on Nov. 24, 1999.

(51) Int. Cl. A61K 48/00; A61K 39/00; C07K 14/705

(52) U.S. Cl. 424/93.21; 424/185.1; 530/350

(57) ABSTRACT

Methods of thymic vaccination are described, in which a polypeptide of interest is administered and which allows positive or negative selection of a T cell receptor (TCR) specificity in the thymus, to retain a desired specificity (positive) or to eliminate an undesired specificity (negative) at the level of TCR repertoire development, in order to generate TCRs which are designed to recognize disease antigens or foreign antigens, such as to treat or prevent cancers, autoimmune diseases, infections, or effects of biological warfare agents.

### RELATED APPLICATIONS

This application is a continuation application of and claims priority to PCT Application No. PCT/US00/31502, filed Nov. 16, 2000, which designates the U.S. and was published in English, which is a Continuation-in-Part of and claims the benefit of U.S. Provisional Application No. 60/168,167, filed Nov. 30, 1999, which is a Continuation-in-Part of and claims the benefit of U.S. Provisional Application No. 60/167,378, filed Nov. 24, 1999, the entire teachings of the above applications are incorporated herein by reference.

### GOVERNMENT SUPPORT

The work described herein was supported in part by Grants AI19807, GM56008, and AI45022 from the National Institutes of Health. The government has certain rights in the invention.

### BACKGROUND OF THE INVENTION

T cell receptors (TCRs) are generated in the thymus through a stochastic process involving rearrangement of V, D and J gene segment elements (Chien et al., Nature, 312:31-35 (1984); Hendrick et al., Nature, 308:149-153 (1984); Saito et al., Nature, 312:36-40 (1984); Yoshikai et al., Nature, 312:521-52 (1984); Davis, M. M. and Bjorkman, P. J., Nature, 334:395-402 (1988) and Fowlkes, B. J. and Pardoll, D. M., Adv. Immunol., 44:207-264 (1989)). Recombinatorial diversity resulting from the joining of the various gene segments and association of diverse α and β subunits coupled with junctional diversity arising from N and P nucleotide additions gives rise to enormous diversity, approximately 10¹⁶ TCR types. Many of these receptor specificities are useful to the organism in establishing protective cognate immune responses. On the other hand, some of the TCRs so created may be detrimental, including ones with self-reactive specificities able to mediate autoimmune diseases. It is the process of negative selection in the thymus that eliminates, in large part, unwanted autoreactive specificities (Nossal, G. J. V., Cell, 76:229-239 (1994)). In addition, cells bearing TCRs unable to productively interact in any manner with the MHC of the host are lost through a death by neglect mechanism. Positive selection enriches for those useful TCRs which are restricted by self-MHC molecules expressed by the individual (Beven, M. J., Nature, 269:417-418 (1977); Berg et al., Cell, 58:1035-1046 (1989); Zinkemagel et al., J. Exp. Med., 147:882-896 (1978); von Boehmer, H., Cell, 76:219-228 (1994); Fowlkes, B. J. and Schweighoffer, E., Curr. Opin. Immunol., 7:188-195 (1995); Jameson et al., Annu. Rev. Immunol., 13:93-126 (1995)). These selection processes shape the T cell repertoire.

### SUMMARY OF THE INVENTION

The present invention is drawn to methods of influencing the selection processes of T cell receptors (TCRs) in order to influence the T cell repertoire of an host. In the methods, a polypeptide of interest or peptidomimetic is administered; the polypeptide of interest or peptidomimetic is a polypeptide that causes selection (either positive or negative) of thymocytes having a T cell receptor specificity in the thymus. The methods of influencing the TCR selection processes can be used for thymic vaccination, which causes selection of thymocytes with TCR specificities that are designed to recognize disease antigens or foreign antigens, such as to treat or prevent cancers, autoimmune diseases, infections, or effects of biological warfare agents. Polypeptides, vaccine compositions, expanded thymic cell populations produced according to the methods are described herein. Also described is a synthetic thymus comprising stromal elements bearing relevant MHC molecules and loaded with the desired polypeptides suitable for carrying out the invention.

### BRIEF DESCRIPTION OF THE DRAWINGS

The foregoing and other objects, features and advantages of the invention will be apparent from the following more particular description of preferred embodiments of the invention.

FIG. 1 depicts the proliferation of splenic T cells to VSVS and p4-substituted VSV8 variant peptides.

FIGS. 2A-2B demonstrate that L4, norvaline4, and γ-methylleucine4 induce positive selection of N15tg RAG-2^{-/-}β₂M^{-/-} thymocytes phenotypically and functionally. FIG. 2A). The total number of CD8⁺ SP thymocytes (mean±SD of four different lobes) after FTOC are shown for the indicated culture conditions. FIG. 2B). Thymocytes selected on L4, norvaline4 and γ-methylleucine4 are functionally responsive to VSV8.

### DETAILED DESCRIPTION OF THE INVENTION

A description of preferred embodiments of the invention follows.

The invention is based on the discovery that the T cell repertoire of an individual can be influenced by targeting the selection processes of TCRs. As described herein, N15 TCR transgenic (tg) RAG-2^{-/-}H-2b mice recognizing the vesicular stomatitis virus (VSV8) octapeptide RGYVYQGL bound to Kb were utilized in conjunction with VSV8 variants differing only at the central p4 position to probe the specificity of TCR selection. The V4I mutant octamer, like VSV8, induces negative selection of immature double positive (DP) thymocytes on the p₂M^{+/+} background and is a strong agonist for mature N15 T cells. In contrast, V4L or V4 norvaline octamers promote positive selection in N15 tg β₂M^{-/-}RAG-2^{-/-}H-2^{b} FTOC and are weak agonists for N15 T cells. Hence, the absence of a p4 side chain β-methyl group results in positive selection of the N15 TCR. Hydrophobicity of the p4 residues also modulates thymocyte fate: the positively selecting norvaline and leucine variants have one and two Cγ-methyl groups, respectively, while the weakly selecting γ-methylleucine p4 contains three Cγ-methyl groups. Moreover, the most hydrophobic octamer containing a p4 cyclohexylglycine substitution fails to select. Thus, for N15 and other class I MHC-restricted TCRs, there is a high degree of structural specificity to peptide-dependent thymic selection processes.

In addition, Applicants have determined that this structural specificity also applies to Class II MHC-restricted TCRs. As described herein, the crystal structure of a complex involving the D10 T cell receptor (TCR), 16 residue foreign peptide antigen and the I-A^{k} self-MHC class II molecule has been assessed at 3.2 Å resolution. The D10 TCR is oriented in an orthogonal mode relative to its peptide-MHC (pMHC) ligand, necessitated by the amino-terminal extension of peptide residues projecting from the MHC class II antigen-binding groove as part of a mini β-sheet. Consequently, the disposition of D10 CDR loops is altered relative to that of most pMHCI-specific TCRs; the latter TCRs assume a diagonal orientation although with substantial variability. Peptide recognition involves P-1 to P8 residues, being dominated by the Vα domain which also binds to the class II MHC β₁ helix. The fact that docking is limited to one segment of MHC-bound peptide explains the epitope recognition and altered peptide ligand effects, provides a structural basis for alloreactivity, and illustrates how bacterial superantigens can span the TCR-pMHCII surface. Furthermore, the opposing processes of positive and negative selection that are present in the T lineage repertoire can be probed by utilizing octapeptides designed based upon the limited segment of the MHC-bound peptide that is involved in docking.

As a result of these discoveries, methods are now available to influence the selection processes of thymocytes bearing TCRs (both Class I and Class II MHC restricted), and thereby to perform thymic vaccination. The term, “selection process” refers to positive or negative selection of thymocytes with a targeted TCR specificity in the thymus, to retain a desired specificity (positive) or to eliminate an undesired specificity (negative). “Thymic vaccination” refers to administration of a polypeptide which influences the selection processes of TCRs while still in the thymus, thereby altering cognate antigens in order to create variants which positively select desired TCR specificities at the level of repertoire development, or which negatively select undesired TCR specificities at the level of repertoire development.

The invention provides a method for educating thymic cells to recognize disease or foreign antigens not previously recognized by the immune system (naive preselected double positive (CD4⁺ CD8⁺) thymic cells). According to the method, naive thymic cells (functional thymus or synthetic thymus) are contacted with a polypeptide of interest that causes selection of a thymocytes with TCR receptor specificities in the thymus capable of recognizing disease antigens or foreign antigens. The so produced “educated” thymic cells can then be induced to leave the thymus and be expanded into a population of antigen recognizing thymic cells that is sufficient to elicit an immune response against the disease antigens or foreign antigens. The methods described herein are applicable to both Class I and Class II MHC complex formation. In general, the antigens can be components such as bacterial, viruses and macro components of cells and soluble antigens such as proteins, peptides, glycoproteins and carbohydrates. Antigens of particular interest are viral or bacterial antigens, allergens, tumor-associated antigens, oncogene products, parasite antigens, fungal antigens or fragments of these. Thus, the peptides and methods described herein can be used to treat cancer tumors and infections in an individual such as, but not limited to, infections caused by bacteria, viruses, fungus and parasites. Examples of human bacterial pathogens include, but are not limited to, Haemophilus influenzae, Escherichia coli, Neisseria meningitidis, Streptococcus pneumoniae, Streptococcus pyogenes, Branhamella catarrhalis, Vibrio cholerae, Corynebacteria diphtheriae, Neisseria gonorrhoeae, Bordetella pertussis, Pseudomonas aeruginosa, Staphylococcus aureus, Klebsiella pneumoniae and Clostridium tetani. Examples of pathogenic virus include, but are not limited to, human immunodeficiency virus, human T cell leukemia virus, respiratory syncyctial virus, hepatitis A, hepatitis B, hepatitis C, non-A and non-B hepatitis virus, herpes simplex virus (types I and II), cytomegalovirus, influenza virus, parainfluenza virus, poliovirus, rotavirus, coronavirus, rubella virus, measles virus, varicella, Epstein Barr virus, adenovirus, papilloma virus and yellow fever virus. Examples of fungal pathogens and opportunistic fungi include species of Cryptococcus, Candida, Coccidioides, Histoplasma, Blastomyces, Sporothrix and Aspergillus.

The methods are particularly useful in vaccinating an individual that has a genetic predisposition to disease and diseases caused by immune deficiency. Thymic cells can be educated to distinguish between antigens of normal cells which are to be left unharmed and those of unwanted intruder which are to be destroyed. For example, a newborn can be genetically screened and if found to be at risk for a certain disease, the newborn's repertoire of T cells can be altered to increase those that are capable of recognizing disease associated antigens. The educated T cells can be induced to come out of the thymus by giving the infant a variation of the polypeptide recognizable by the educated T cells which the immune system could be tricked into recognizing as normal or self.

In one embodiment of the invention, a polypeptide of interest or peptidomimetic is administered to an individual to eliminate (e.g., destroy) thymic cells having TCRs of undesired or detrimental specificity (negative selection). For example, TCRs having self reactive specificities able to mediate autoimmune diseases can be eliminated in the thymus before they leave and induce an autoimmune response. According to this embodiment, thymic cells having TCRs of undesired or detrimental specificity are contacted with polypeptides of interest or peptidomimetics that can reduce or eliminate cells bearing TCRs capable of recognizing a self antigen. Once eliminated the detrimental TCRs are not capable of leaving the thymus and mediating autoimmune disease. Thus, the invention can be used to treat or prevent individuals predisposed to autoimmune diseases or inflammatory diseases. Examples of acute and chronic immune and autoimmune diseases include, but are not limited to, chronic hepatitis, systemic lupus erythematosus, arthritis, thyroidosis, Scleroderma, diabetes mellitus, Graves' disease, Beshet's disease and graft versus host disease (graft rejection).

In the methods of the invention, a polypeptide or peptidomimetic is administered to a host. The host can be any mammal; in a preferred embodiment, the host is a human. The host can be adult, child, infant or fetus. In a preferred embodiment, the host is an infant, because T cells are produced in the thymus gland during the first year of life, and during that time are “educated” to distinguish between antigens of normal cells, which are to be left unharmed, and those of unwanted intruders, which are to be destroyed. When the host is a child or adult, the methods of the invention are carried out using a synthetic thymus in which bone marrow progenitor cells of the host are induced in an implanted thymus to produce thymocytes. An artificial or synthetic thymus can be implanted (e.g., intramuscularly, subcutaneously) into the host and comprises stromal elements bearing relevant MHC molecules and loaded with the desired peptides or peptidomimetic described herein.

The polypeptide of interest is a short polypeptide, having between approximately 6 and 16 amino acids, preferably between 8 and 12 amino acids. The polypeptide of interest is a polypeptide which influences the TCR selection process in the thymus to select TCR specificities (either positive or negative). The polypeptide can include natural amino acids, artificially created amino acids, and/or amino acid analogs, and can also be modified, such as by substituted linkages, glycosylations, acetylations, carboxylations, phosphorylations, ubiquitination, labeling (e.g., with radionuclides), enzymatic modifications, or other modifications known in the art, both naturally and non-naturally occurring. If desired, a carrier molecule, such as another polypeptide or other agent, can be used in conjunction with the polypeptide of interest.

Polypeptides useful for methods described herein are generated by structurally altering the central recognition position of the peptide, i.e., p4 for Class I MHC-restricted TCRs and p5 for Class II MHC-restricted TCRs, as described herein. For example, the VSV8 peptide (RGYVYQGL) (SEQ. ID NO:1) and variants having other amino acids at position 4 (e.g., isoleucine, leucine, norvaline, γ-methylleucine or cyclohexylglycine) can be used for Class I MHC-restricted TCRs. However, other TCR contact residues of the peptide can be altered. Polypeptides used herein can be isolated from naturally-occurring sources, chemically synthesized or recombinantly produced. For example, a nucleic acid molecule can be used to produce a recombinant form of the encoded polypeptide via microbial or eukaryotic cellular processes. Ligating the polynucleotide sequence into a gene construct, such as an expression vector, and transforming or transfecting into hosts, either eukaryotic (yeast, avian, insect, plant or mammalian) or prokaryotic (bacterial cells), are standard procedures used in producing other well known proteins. Similar procedures, or modifications thereof, can be employed to prepare recombinant polypeptides by microbial means or tissue-culture technology. The polypeptides can be isolated or purified (e.g., to homogeneity) from cell culture by a variety of processes. These include, but are not limited to, anion or cation exchange chromatography, ethanol precipitation, affinity chromatography and high performance liquid chromatography (HPLC). The particular method used will depend upon the properties of the polypeptide; appropriate methods will be readily apparent to those skilled in the art. For example, with respect to protein or polypeptide identification, bands identified by gel analysis can be isolated and purified by HPLC, and the resulting purified protein can be sequenced. Alternatively, the purified polypeptide can be enzymatically digested by methods known in the art to produce polypeptide fragments which can be sequenced. The sequencing can be performed, for example, by the methods of Wilm et al. (Nature 379(6564):466-469 (1996)). The polypeptide may be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990).

The sequence of the polypeptide can be determined, for example, by assessing the foreign peptide antigen that binds in the complex of a T cell receptor, antigen and MHC Class I or Class II molecule, such as by examining the crystal structure, as described below. The crystal structure indicates which amino acids of the polypeptide interact with the TCR. Those amino acids of the polypeptide which interact with TCR are referred to herein as “active amino acids,” and their position in the polypeptide (e.g., as amino acid #1, #4, #5 or #6) is referred to as an “active amino acid position”. Varying the type of amino acid at the active amino acid position by substituting the original amino acid with other amino acids, allows determination of what type of amino acids contribute to positive or negative selection of desired TCR specificities. The ability of a polypeptide of interest to stimulate proliferation of the desired TCR cells can be assessed by culturing splenocytes with samples of the polypeptide of interest and assessing activation of the T cells, as described below. The ability of a polypeptide of interest to interact with TCRs on thymocytes can be assessed by performing thymocyte dulling assays, as described below. To assess the ability of a polypeptide of interest to influence TCR negative specificity, an assay can be performed, such as an in vivo assay as described below in which a host is inoculated with the peptide of interest, and the surviving subsets of thymocytes examined after an appropriate incubation time. To assess the ability of a polypeptide of interest to influence positive specificity, an assay can be performed, for example, using fetal thymic organ culture as described below in which the organ is cultured with a sample of the polypeptide of interest, and the type of thymocytes produced are examined after an appropriate incubation time. As described herein, a mouse recipient has been used to exemplify the invention. Similarly, a mouse model having a humanized immune system can be used to appropriately predict human therapies.

Alternatively, a peptidomimetic having the same characteristics as a polypeptide of interest (e.g., the peptidomimetic can influence the selection processes of TCRs in the thymus, altering cognate antigens and creating variants which positively select desired TCR specificities or which negatively select undesired TCR specificities) can be used. The peptidomimetic can be, for example, a complex carbohydrate or other oligomer of individual units or monomers which binds specifically to its binding partner (e.g., the TCR). Peptidomimetics can be developed, for example, with the aid of computerized molecular modeling (see e.g., Fauchere, J. Adv. Drug Res., 15:29 (1986); Veber and Freidinger, TINS 392 (1985); and Evans et al., J. Med. Chem., 30:1229 (1987)). Peptidomimetics that are structurally similar to the peptides described herein can be used to produce an equivalent therapeutic effect. Generally, peptiomimetics are structurally similar to a paradigm peptide (i.e., a peptide that has a biological or pharmacological activity), such as a peptide of interest herein, but have one or more peptide linkages optionally replaced by other organic linkages. Peptidomimetics may be generated, for example, by methods described in Spatola, A. F. in CHEMISTRY AND BIOCHEMISTRY OF AMINO ACIDS, PEPTIDES, AND PROTEINS 267 (B. Weinstein, eds. 1983); Spatola, A. F., Vega Data Vol. 1, Issue 3, “Peptide Backbone Modifications” (March 1983)(general review); Moreley, J. S., Trends Pharm. Sci., pp. 463-468 (1980)(general review); Hudson, D. et al., Int. J. Pept. Prot. Res. 14:177-185 (1979); Spatola, A. F. et al., Life Sci. 38:1243-1249 (1986); Hann, M., J. Chem. Soc. Perkin Trans. I 307-314 (1982); Alnquist, R. G. et al., J. Med. Chem. (1980) 23:1392-1398; Jennings-White, C. et al., Tetrahedron Lett. 23:2533 (1982); Szelke, M. et al., European Appln. EP 45665 (1982) CA:97:39405 (1982); Holladay, M. W. et al., Tetrahedron Lett. 24:4401-4404 (1983); and Hruby, V. J., Life Sci. 31:189-199 (1982).

To perform the methods of the invention, a polypeptide of interest or peptidomimetic, which influences the selection processes of thymocytes with TCRs of targeted specificities, is administered to the host animal. The polypeptide of interest or peptidomimetic is administered by a means which exposes it to the immune system in the host animal. In a preferred embodiment, the polypeptide of interest can be administered in a pharmaceutical composition. For instance, a polypeptide or peptidomimetic can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyro-lidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrol-lidone, sodium saccharine, cellulose, magnesium carbonate, etc.

Methods of introduction of these compositions include, but are not limited to, intradermal, intramuscular, intraperitoneal, intraocular, intravenous, subcutaneous, oral and intranasal. Other suitable methods of introduction can also include gene therapy, rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water, saline or dextrose/water. Where the composition is administered by injection, an ampoule of sterile water for injection or saline can be provided so that the ingredients may be mixed prior to administration.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in an effective amount. The amount of agents which will be effective in the generation of desired TCRs can be determined by standard clinical techniques. In addition, in vitro or in vivo assays may optionally be employed to help identify optimal dosage ranges. The precise dose to be employed in the formulation will also depend on the route of administration and should be decided according to the judgment of an individual of ordinary skill in the art. Effective doses may be extrapolated from dose-response curves derived from in vitro or animal model test systems.

Administration of the polypeptide of interest or peptidomimetic alters TCRs with specificity for cognate antigens and thereby positively selects desired TCR specificities at the level of repertoire development, or which negatively select undesired TCR specificities at the level of repertoire development. This alteration, also referred to as “thymic vaccination,” can be used to generate specific reactivities in the thymus, in order to generate TCRs which are designed to recognize disease antigens or foreign antigens, such as to treat or prevent cancers, autoimmune diseases, infections, or effects of biological warfare agents. For example, if an individual was at risk for a certain disease (e.g., childhood leukemia), a polypeptide of interest or peptidomimetic that generates positive selection for TCRs that are capable of recognizing cells affected by the disease can be administered. Alternatively, if an individual was at risk for, or affected by, an autoimmune disease, a polypeptide of interest or peptidomimetic that generates negative selection of “self” antigens can be administered, so that TCRs that recognize the self antigen are reduced or eliminated.

The invention also provides a pharmaceutical pack or kit comprising one or more containers filled with one or more of the ingredients (e.g., the polypeptides of interest or peptidomimetics) of the pharmaceutical compositions described herein. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

The invention is further illustrated by the following references, which are not intended to be limiting in any way. The teachings of all references cited herein are incorporated by reference in their entirety.

### EXAMPLES

The examples set forth below describe in detail many diagrams of molecular structures and interactions. Color photographs of the diagrams can be found in the following two references which describe Applicants' work and which were published after the filing date of the priority applications: Sasada, T. et al., Eur. J. Immunol. 30(5):1281-9 (May 2000), and in Reinherz, E. L. et al., Science 286 (5446):1913-21 (December 1999). The entire teachings of these references, and the references cited in the Specification, are incorporated herein by reference.

### Example 1

### Influence of Subtle Structural Variation Involving the p4 Residue of an MHC Class I-Bound Peptide on Thymic Selection

Recently, using TCR-transgenic (N15tg) β₂-microglobulin deficient (β₂M^{-/-}) RAG-2^{-/-}H-2^{b} mice specific for the VSV8 octapeptide bound to K^{b}, a single weak agonist peptide variant V4L (L4) was identified which induced phenotypic and functional T cell maturation (Ghendler et al., Proc. Natl. Acad. Sci. USA, 95:10061-10066 (1998)). The cognate VSV8 peptide, in contrast, triggered negative selection. The crystal structure of L4/K^{b} was determined and refined to 2.1 Å for comparison with the VSV8/K^{b} structure at similar resolution. Aside from changes on the side chain of the p4 position of L4 and the resulting alteration of the exposed K^{b} Lys66 side chain, these two structures are essentially identical. Hence, focal, local structural change in the pMHC can be readily discerned by the TCR.

An objective of the work described herein was to define a structure-activity relationship between alteration of the p4 side chain constituent and thymic selection. The findings suggested that the survival outcome for thymocyte-bearing the N15 TCR can be reduced to simple chemical parameters involving the selecting peptide. Similar rules apply to at least some other class I MHC-restricted TCRs.

Experimental Procedures

Mice: N15 TCR tg (N15^{+/+}) RAG^{-/-} (H-2^{b}) and N15 TCR tg (N15^{+/+}) RAG^{-/-}β₂M^{-/-} mice were generated as previously described (Ghendler et al., Eur. J. Immunol., 27:2279-2289 (1997); Ghendler et al., J. Exp. Med., 187:1529-1536 (1998)). The lack of RAG-2 or β₂M gene expression in knockout animals were identified based on the fluorescence-activated cell sorter (FACS) analysis on peripheral blood cells (Ghendler et al., Proc. Natl. Acad. Sci. USA, 95:10061-10066 (1998)). The homozygosity of the N15 TCR transgenes was proved by subsequent breeding analysis. TAP^{-/-} (129/Ola X C57BL/6) mice were purchased from Taconic (N.Y.). All lines were maintained and bred under sterile barrier conditions at the animal facility of Dana-Farber Cancer Institute.

Peptides Synthesis

VSV8 variant peptides were synthesized by standard solid phase methods on an Applied Biosystems 430A synthesizer (Foster City, Calif.) at the Biopolymers Laboratory of Massachusetts Institute of Technology. Norvaline (nV) and γ-methylleucine (mL) were obtained from Bachem Biosicence Inc. (King of Prussia, Pa.) and cyclohexylglycine (chG) was from Sigma (St. Louis, Mo.). All peptides were purified by reverse phase HPLC (Hewlett Packard HPLC 1100, Palo Alto, Calif.) with a C4, 2 mm column. Peptides were analyzed for purity and correct molecular weight by electrospray mass spectrometry, amino acid analysis and HPLC. Peptides are named to indicate the substituted amino acid and the position in the sequence (e.g., 14 denotes replacement of valine by isoleucine at the fourth residue of the VSV8 peptide).

Antibodies and Flow Cytometric Analysis

The following mAbs were used: R-phycoerythrin (PE) anti-mouse CD4 (H129.19; Life Technologies, Grand Island, N.Y.); Cychrome anti-mouse CD8α (53-6.7; Pharmingen, San Diego, Calif.). For flow cytometry, single cell thyrnocyte suspensions were prepared in phosphate-buffered saline (PBS) containing 2% fetal calf serum (FCS). Thymocytes were stained at 5×10⁶ cells per ml in PBS-2% FCS containing the antibodies at saturating concentrations. Phenotypes and proportions of thymocyte subsets were analyzed by two-color flow cytometry using FACScan (Beckton Dickinson) and the Cell Quest program. Dead cells were excluded from the analysis by forward and side scatter gating.

DP Dulling Assay

Peritoneal exudate cells (PEC) from TAP^{-/-} (129/Ola X C57BL/6) mice (Taconic), induced 5 days previously with 2 ml of 3% thioglycollate, were suspended in AIM-V medium (Life Technologies) and plated at 1×10⁶ per well in a 96-well microtiter plate. After adherence for 2 hr, monolayers were washed with AIM-V medium four times. Thymocytes (5×10⁵) from 4- to 6-week-old N15 tg RAG^{-/-}/β₂M^{-/-} were co-cultured with the PEC for 18 hr at 37° C., and stained for the expression of CD4 and CD8α.

Peptide Injection

Negative selection was examined by the reduction of CD4⁺CD8⁺DP thymocytes in N15 tg RAG^{-/-}β₂M^{-/-}H-2^{b} mice after injection of peptides as described (Ghendler et al., Eur. J. Immunol., 27:2279-2289 (1997); Ghendler et al., J. Exp. Med., 187:1529-1536 (1998)). Twenty-four μg of each peptide dissolved in 200 ml PBS was injected into the tail vein of 4-week-old N15tg RAG-2^{-/-} mice. After 24 hr of peptide injection, thymocytes were stained for the expression of CD4 and CD8α.

Fetal Thymic Organ Culture (FTOC)

Fetuses of N15 tg RAG^{-/-}β₂M^{-/-}H-₂^{b} mice were dissected at day 15.5 (plug=day 1) and fetal thymic lobes were cultured with or without the indicated peptides as described (Clayton et al., EMBO J, 16:2282-2293 (1997)). Human β₂M (Calbiochem, San Diego, Calif.) was added to AIM-V medium at 5 μg/ml, and the medium was replaced every 48 h. After 7 days, thymocytes were stained for the expression of CD4 and CD8α, or were tested for their capacity to respond to antigen in a 2 day proliferation assay, as described below.

Proliferation Assay

Thymocytes from the organ cultures or fresh splenocytes from N15 tg RAG-2^{-/-}H-2^{b} mice (1×10⁵/well) were incubated at 37° C. with 2×10⁴ irradiated EL-4 cells, which were pre-loaded for 2 h with 1 nM or 10 nM VSV8 in the presence of 100 U/ml recombinant IL-2 in AIM-V medium containing 50 μM 2-ME. After 48 h of incubation, 0.4 μCi per well of ³H-TdR (ICN Biomedicals, Aurora, Ohio) was added, and after an additional 18 h of culture at 37° C., the cells were harvested and the incorporated radioactivity was measured.

### RESULTS AND DISCUSSION

p4 Variants of the VSV8 Octapeptide

In the B6 mouse, the VSV8 octapeptide bound to the Kb MHC class I molecule is a major determinant of the vesicular stomatitis virus nuclear protein recognized by CD8 CTL. When VSV8 is in complex with K^{b}, only p1 Arg, p4 Val and p6 Gln peptide side chains point to solvent. Hence, three VSV8 residues can make contact with the TCR (Ghendler et al, Proc. Natl. Acad. Sci. USA, 95:10061-10066 (1998)). An examination of the structure of the VSV8 octamer bound to H-2K^{b} and variant p4 side chains shows upward pointing TCR contact p1R, p4V and p6Q residues. Substitutions at any of these positions dramatically and adversely affect T cell recognition as judged by cytolytic effector function (Ghendler et al., Proc. Natl. Acad. Sci. USA, 95:10061-10066 (1998)) or other functional measurements of T cell activation (Ghendler et al., Proc. Natl. Acad. Sci. USA, 95:10061-10066 (1998)). The effects of alterations at the p4 position on thymic selection were studied in detail since this residue is centrally located on the recognition surface of the VSV8 K^{b} complex when bound to the N15 TCR (Ghendler et al., Proc. Natl. Acad. Sci. USA, 95:10061-10066 (1998)). Moreover, earlier studies showed that even a conservative substitution, namely p4 Val Leu, altered the fate of DP thymocytes, leading to positive selection in contrast to negative selection resulting from in vivo injection of N15 tg Rag-2^{-/-}H-2^{b} mice with the cognate peptide (Ghendler et al., Proc. Natl. Acad. Sci. USA, 95:10061-10066 (1998)).

In the present study, a series of altered peptide ligands (APL) of VSV8 were created, harboring highly related yet distinct amino acid substitutions at the p4 position. The amino acids used for the substitutions included valine, isoleucine, leucine, norvaline, gamma-methylleneine, and cyclohexylglycine. The structure of the side chains of these substitutions varied, relative to the valine R group at the p4 position of VSV8. In particular, for example, the Isoleucine4 variant (14), like the p4 Val in VSV8, has a Cβ branch (methyl group) whereas the Leucine4 (L4) variant does not. The hydrophobicity of I4 and L4 are identical, however. Norvaline4, L4 and γ-methylleucine4 all lack a Cβ methyl group but differ in having 1, 2 and 3 Cγ-methyl groups, respectively. Cyclohexylglycine4 is the most hydrophobic of all p4 side chain R groups. Given that the p3, p5 and p8 anchor residues have not been modified in these APL, it is not surprising that the Kb binding affinity for these variants relative to VSV8 are unaltered (Ghendler et al., Proc. Natl. Acad. Sci. USA, 95:10061-10066 (1998)).

Activation of Mature N15 Splenic CD8⁺ T Cells by p4 Variants

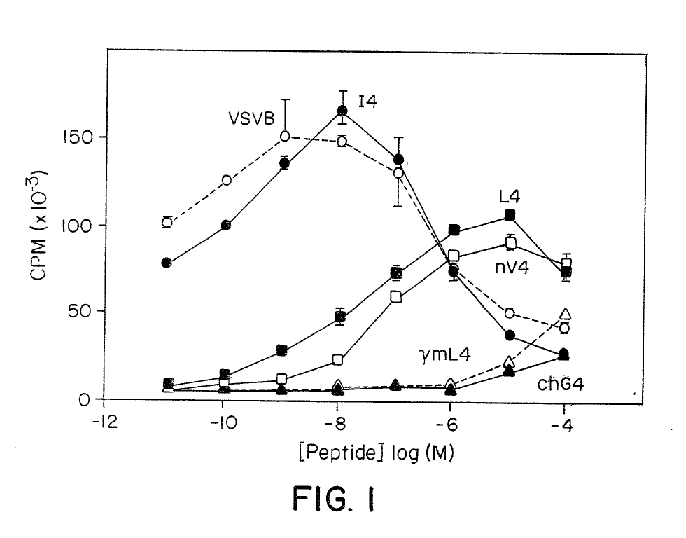

To examine the ability of APL to stimulate proliferation of CD8+ N15 TCR-bearing T cells, splenocytes (1×10⁵) from N15 tg Rag-2^{-/-}H-2^{b} mice were cultured with varying molar concentrations of the individual peptides using irradiated EL4 cells as K^{b}-bearing APC (2×10⁴). After 48 h of stimulation, cells were pulsed with ³H-TdR and mean±SD of triplicate cultures determined. As shown in FIG. 1, both VSV8 and I4 maximally stimulate ³H-TdR incorporation by 10⁻⁸ M. In contrast, in norvaline4 and L4 maximal stimulation requires a 10⁻⁵ M peptide concentration. Thus, norvaline4 and L4 are weak agonists, differing by ≧1000 fold from I4 and VSV8. γ-methylleucine4 and cyclohexylglycine4 are even weaker with detectable stimulating activity present only at a peptide concentration of 10⁻⁴ M.

Interaction of the N15 TCR with VSV8 and APL Detected by DP Thymocyte Dulling Assay

To determine which of these APL when complexed to cell surface bound Kb is able to interact with the N15 TCRs on immature thymocytes, a thymocyte dulling assay was performed. For this purpose, N15 tg Rag-2^{-/-}β2M^{-/-} thymocytes were cultured in vitro for 18 h with peritoneal exudate cells (PEC) from TAP^{-/-} mice at varying peptide concentrations. In this assay (Bamden et al., Eur. J. Immunol., 24:2452-2456 (1994); Hogquist et al., Immunity, 6:389-399 (1997); Vasquez et al, J. Exp. Med., 175:1307-1316 (1992)), TCR interaction with pMHC ligand is detected as a reduction in the intensity of CD4 and CD8 expression on the surface of the DP thymocytes. Alterations in the expression of CD4 and CD8α on DP thymocytes were detected by flow cytometry after gating on 10,000 live cells. Thus, the DP dulling assay results detected the interactions of N15tg thyrnocytes with VSV8 and altered peptide ligands of VSV8. A negative control was used that contained thymocytes plus PEC cultured in the absence of any exogenous peptide additions; for the negative control, in the absence of peptide, 90% of the cells are DP thymocytes whose CD4 and CD8 expression falls in an expected range. In contrast, in the VSV8 exposed cultures, only 10-26% of thymocytes fall in this range at peptide concentrations from 10 μM to 1 nM. The remaining thymocytes have clearly reduced co-receptor expression levels. Even at a VSV8 concentration of 10 μM, there is dulling of a fraction (˜5%) of DP thymocytes. An identical result was observed with I4. Dulling is also observed with L4 at 10 μM and 100 nM concentrations. With norvaline4, dulling is seen at 10 μM and minimally at 100 nM whereas with γ-methylleucine4 or cyclohexylglycine4, activity is only observed at a peptide concentration of 10 mM. Thus, interaction is observed with all VSV8-related peptides. The potency of the dulling effects vary with the individual peptides, roughly corresponding to the strength of agonist activity observed with the mature peripheral T cells, hence VSV8>L4/norvaline4>γ-methylleucine4/cyclohexylglycine4.

Negative Selection of N15 TCR-Bearing DP Thymocytes by Octamers with a p4 Residue Containing a Cb Methyl Group

While the above dulling assay offers a sensitive means to detect TCR-pMHC interaction involving thymocytes and APCs, it does not provide information about the ability of peptides to mediate positive vs. negative selection. To examine the capacity of the various peptides to induce negative selection of DP thymocytes, an in vivo assay was performed. Individual N15 tg Rag-2^{-/-}H-2^{b} mice were injected i.v. with 24 μg of each peptide and the surviving subsets of thymocytes examined after 24 h. The expression of CD4 and CD8α in thymocytes were detected by two-color flow cytometry after gating on live cells. Without injection, the % of DP thymocytes is 81. This number is virtually the same for L4, norvaline4, γ-methylleucine4 and cyclohexylglycine4 (64, 72, 75 and 84%, respectively). By contrast, with VSV8 and 14, the % of DP thymocytes was diminished (14 and 18, respectively). Prior studies showed that the majority of DP thymocytes were deleted by a caspase-dependent apoptotic mechanism during this time (Ghendler et al., J. Exp. Med., 187:1529-1536 (1998); Clayton et al, EMBO J., 16:2282-2293 (1997)). Consistent with this observation, <10% of the surviving thymocytes were DP in VSV8 injected animals compared with 81% in uninjected animals. A similar deletion was observed following administration of the I4 peptide. In contrast, L4, norvaline4, γ-methylleucine4 and cyclohexylglycine4 induced no deletion. Careful titration analysis of varying molar concentrations of each peptide in FTOC failed to detect significant reduction in DP thymocytes at any concentration of L4, norvaline 4, γ-methyleucine4 and cyclohexylglycine4 tested. In view of the chemical differences among these VSV8 variants, it would appear that a Cβ methyl group needs to be present on the p4 side chain side chain to induce negative selection.

Positive Selection by Octamers Lacking a p4 C, Methyl Group and Further Modulation by Side Chain Hydrophobicity

To investigate which other APL in addition to L4 might induce positive selection, FTOC was performed with N15 tg Rag-2^{-/-}β₂M^{-/-} thymus lobes using synthetic media and human β₂M with and without VSV8 or other individual APL. FTOC was performed by using N15tg RAG-2^{-/-}β₂M_{-/-} thymic lobes in AIM-V medium containing 5 mg/ml human β₂M with or without the indicated peptides (VSV8, I4, L4: 10 μM; norvaline4, γ-methylleucine4, Cyclohexylglycine4: 100 μM). After 7 days, thymocytes were released from the lobes by passing through a steel mesh and cell numbers were counted by flow cytometry to detect CD4 versus CD8α staining profiles of total thymocytes after FTOC. With VSV8 in I4, only 1% of CD8 SP thymocytes were generated compared to 9% for the control culture (minus peptide). With 100 μM L4 and norvaline4, 57% and 63%, respectively, of thymocytes were CD8⁺ after 7 days of culture. Positive selection was also detected using 10 μM concentrations of these two peptides. Although some positive selection was also induced by 100 μM γ-methylleucine, no increase in positive selection was observed at a 10 μM peptide concentration.

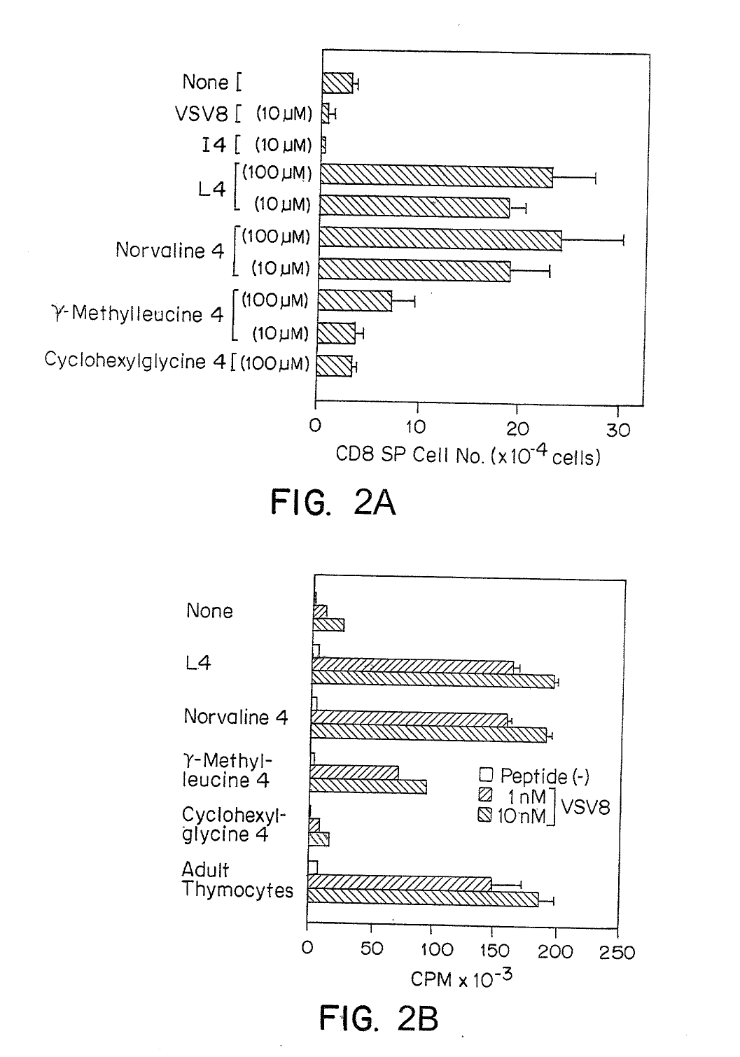

Moreover, positive selection was not augmented by cyclohexylglycine4 even at a 100 μM peptide concentration. FIG. 2A offers information on the absolute number of surviving CD8 SP thymocytes generated. The total number of CD8⁺ SP thymocytes (mean±SD of four different lobes) after FTOC are shown for the indicated culture conditions. The numbers of CD8 SP thymocytes were calculated by quantifying the total numbers of thymocytes and percentages of CD8⁺ SP subsets determined above by FACS. Number of CD8 SP thymocytes positive selected by L4 and norvaline4 are quite comparable and each ˜10 fold more than the “peptide minus” control.

Perhaps more importantly, thymocytes from the above organ cultures or fresh thymocytes from an adult N15tg RAG-2^{-/-} (H-2b) mice (1×10⁵/well) were assayed for their proliferative response to 2×10⁴ irradiated EL-4 cells, in the presence of 1 nM or 10 nM VSV8 or no peptide. [³H] thymidine incorporation was determined after 48 hr. Results are shown as mean±SD of triplicate cultures in FIG. 2B, which demonstrates that the phenotypic increase in CD8 SP thymocytes induced by L4 and norvaline4 and, to a lesser extent, by γ-methylleucine4, is accompanied by functional maturation.

Hence, if FTOC are established in the presence of L4 or norvaline4 for 7 days, the subsequent immune response of the harvested thymocytes to the VSV8 cognate peptide is dramatically increased as judged by cellular proliferation of VSV8 at 1 or 10 nM. In fact, the proliferation of thymocytes obtained from the L4 or norvaline4 culture at FTOCs is comparable to that of adult N15 tg Rag-2^{-/-}H-2^{b} thymocyte controls. By contrast, little proliferation is observed to VSV8 by thymocytes harvested from FTOC lacking exogenous peptide addition. Consistent with the data discussed above, moreover, exposure of FTOC to γ-methylleucine4 for 7 days augments subsequent proliferation of fetal thymocytes to VSV8 whereas comparable culture with cyclohexylglycine4 is without effect.

Implications

Recently, three experimental approaches toward characterization of the role of peptides in positive selection have been utilized: 1) analysis of FTOC in non-TCR tg or TCR tg mouse strains carrying mutations that interfere with peptide loading and surface expression of class I MHC molecules (β₂M^{-/-}, TAP1^{-/-}) (Ashton-Rickardt et al., Cell, 76:651-663 (1994); Hogquist et al, J. Exp. Med., 177:1469-1473 (1993); Hogquist et a.l., Cell, 76:17-27 (1994); Hogquist et al., Immunity, 3:79-86 (1995); 2) analysis of the T cell repertoire of class II-restricted responses in mice expressing a single pMHC class II complex generated from a transgenic construct of class II β chain covalently linked to a peptide, or in H-2M null mutant mice where class II molecules are filled with an invariant chain (Ii) peptide (Ignatowicz et al., Cell, 84:521-529 (1996); Ignatowicz et al., Immunity, 7:179-186 (1997); Tourne et al, Immunity, 7:187-195 (1997); Surh et al., Immunity, 7:209-219 (1997); Grubin et al., Immunity, 7:197-208 (1997); or 3) analysis of T cell responses in mice intrathymically infected by an adenoviral-based vector-mediated delivery of invariant chain-peptide fusion proteins (Nakano et al., Science, 275:678-683 (1997)). From such studies, it is clear that peptides are essential to foster positive selection, that peptides bound to a given MHC allele mediate positive selection in a manner which is quite specific for a given TCR but that a single pMHC complex can facilitate differentiation of a substantial number of TCRs. For example, in non-tg β₂M^{-/-} FTOC, the size of the selected CD8 SP repertoire increases with the greater complexity of exposed peptides. Analysis of FTOC for TCR tg TAP1^{-/-} and TCR β₂M^{-/-} is also consistent with the role for peptides in induction of positive selection. Although initial results suggested that in the case of the TCR tg β₂M^{-/-} model, antagonistic peptides induced positive selection while the cognate peptide induced negative selection, the relationship between agonist and antagonist vis-a-vis selection has become less clear (Spain et al., Immunol., 152:1709-1717 (1994); Page et al., Proc. Natl. Acad. Sci. USA, 91:4057-4061 (1994); Sebzda etal., J. Exp. Med., 183:1093-1104 (1996)). Other studies, particularly those utilizing TCR tg TAP1^{-/-}FTOC have shown that positive selection can be driven by low concentrations of cognate antigenic peptide whereas negative selection can be induced by high concentrations of the same peptide (Ashton-Rickardt et al., Cell, 76:651-663 (1994)). Moreover, in animals exposed to the adenovirus delivery system, cognate peptide fosters positive selection in a class II MHC-based system (Nakano et al, Science, 275:678-683 (1997)).

For the N15 TCR, no concentration of VSV8 or I4 agonists was observed to induce positive selection in FTOC. The specificity of the selection process is striking. Hence, L4 and I4 VSV8 variant peptides induced positive and negative selection, respectively, despite the fact that all residues except p4 are the same. Moreover, the hydrophobicity of the two p4 substituents is identical. These findings indicate that the β branch of the isoleucine results in negative selection while the absence of the β branch facilitates development, i.e. positive selection. That VSV8 itself induces negative selection of N15 TCR bearing DP thymocytes is consistent with this notion since valine has a Cβ methyl group as well. Crystallographic data further supports this hypothesis. In the case of the L4/K^{b} structure, the aliphatic portion (Cβ and Cγ atoms) of the exposed side chain of Lys66 on the α1 helix of K^{b} swings about 2.5 Å toward the antigen binding groove in the absence of p4 side chain Cβ methyl group (Ghendler et al., Proc. Natl. Acad. Sci. USA, 95:10061-10066 (1998)). By contrast, in VSV8/K^{b} there is van der Waal's contact between the Cγ2 atom of the p4 Val of VSV8 and the Cd atom of Lys66 (Ghendler et al., Proc. Natl. Acad. Sci. USA, 95:10061-10066 (1998)). This interaction helps to “prop up” the Lys66 side chain. Likewise, in the I4/K^{b} structure which has been recently obtained (Ghendler et al., Proc. Natl. Acad. Sci. USA, 95:10061-10066 (1998)) the side chain of I4 makes close contact with the Cδ atom of Lys66. The movement of the Lys66 side chain towards the MHC groove in the L4/K^{b} complex may increase the off-rate of the TCR-pMHC interaction sufficiently to permit the N15 TCR tg DP thymocytes to escape from TCR-triggered negative selection. Conversely, by directing the Lys66 side chain upwards toward the TCR, it is postulated that the p4 β-methyl groups of VSV8 and its I4 variant augment the TCR-pMHC interaction. The latter would facilitate negative selection. Given that the monomeric affinity of the N15 TCR for VSV8/K^{b} is >200 μM, the predictably worse affinity of the N15 TCR for L4/K^{b} is not measurable by existing BIAcore technology.

Another determinant of selection outcome is the hydrophobicity of the p4 side chain. Thus, while norvaline4 and L4 peptides with one and two Cγ-methyl groups, respectively, induced equivalent levels of positive selection, the three Cγ-methyl group containing γ-methylleucine p4 R group was a weak positive selector. The larger and hydrophobic cyclohexylglycine was without any discernible positively selecting effect. As none of these four variants induced negative selection, it seems unlikely that the hydrophobicity enhanced TCR contact. More likely, the increasing side chain bulk serves to reduce interface complementarity, making contacts less favorable for either negative or positive selection.

It seems clear that p1 and p6 residues of the VSV8 octamer participate in the selection process as well. For example, a p1 Arg Lys mutation in VSV8 results in a peptide with neither the ability to negatively or positively select in the N15 TCR tg system (Ghendler et al., Proc. Natl. Acad. Sci. USA, 95:10061-10066 (1998)). Nonetheless, of more than 4 dozen APL variants at p 1, p4 and p6 tested, the L4, norvaline4 and γ-methylleucine4 peptides are the only ones able to induce positive selection. Given these observations and the central position of the p4 residue in the TCR-pMHC interaction surface, these results suggest that alteration at p4 is pivotal for regulating the balance between positive and negative selection. In view of the common docking orientation of TCRs to pMHC1 molecules, moreover, it seems likely that these results are applicable to other class I-restricted TCRs as well.

### Example 2

### Specific Recognition Function of T Lymphocytes

The adaptive immune response is dependent on the specific recognition function of αβ T lymphocytes (1). Each T cell detects a protein fragment (i.e. peptide) of a self-protein or cell-associated pathogen derived from either viral, bacterial, fungal, parasitic or tumor cell origin bound to a major histocompatibility complex (MHC) molecule. The physical binding of the peptide-MHC (pMHC) complex to the TCR then initiates a series of signal transduction events. Once triggered, T lymphocytes release cytotoxic molecules and/or inflammatory cytokines which destroy the infected or otherwise altered cells through various effector mechanisms. For a given αβ T lymphocyte, immune recognition is mediated via a clonotypic αβ heterodimeric structure (Ti) non-covalently associated with the monomorphic CD3 signaling components.

Sequence analysis of TCR αβ heterodimers first suggested that they would share with antibodies a common structure (2); however, direct evidence supporting this notion has been provided only in the last several years, initially from crystal structures of αβ TCR components (3) and subsequently through analysis of intact αβ TCR heterodimers alone (4) or in complex with pMHC (5). As anticipated, aside from the C_{α} domain, the three dimensional structure of the TCR resembles an antibody Fαβ fragment such that each of the α and β chains consists of canonical immunoglobulin (Ig)-like variable and constant domains with the hypervariable complementarity-determining regions (CDRs) from the two variable domains (Vα and Vβ) forming the ligand binding site for pMHC within the immunorecognition module.

Class I and class II MHC molecules have evolved to facilitate T cell detection of pathogens residing in distinct intracellular compartments (6-8). Although the domain organization of the class I and class II MHC extracellular segments is different, these molecules possess a very similar overall antigen presenting groove consisting of α1 plus α2 domains and α1 plus β1 domains for class I and class II MHC, respectively (9-11). For both molecules, the α-helices of these two domains form the sides of the antigen binding groove with the floor created by an eight-stranded β-sheet arising from both domains. However, unique structural features of the two MHC classes dictate the binding of peptides differing in length and composition (reviewed in 12). The bipartite nature of the immune recognition molecules expressed on antigen presenting cells is reflected at the level of αβ T lymphocytes by the evolution of two subsets bearing specialized MHC binding structures, termed CD4 and CD8 (13, 14). CD8 cells are cytolytic precursor/effector cells, whereas CD4 cells comprise the helper T cell subset which initiates inflammatory responses. CD4 and CD8 molecules have been termed co-receptors since CD4 binds to the membrane proximal β2 domain of class II MHC, while CD8 (αα and αβ) isoforms bind to the corresponding α3 domain of class I MHC (15, 16).

At present, four distinct class I-restricted TCRs have been crystallized in complex with their specific pMHCl ligands (5). Rather extensive interactions with the pMHC α-helices has suggested a common “diagonal” docking mode, regardless of TCR specificity or species origin, in which the TCR Vα domain overlies the class I MHC α2 helix and the Vβ domain overlies the MHC α1 helix. As a result, the CDR1 and CDR3 loops of the TCR Vα and Vβ domains make the major contacts with the peptide while the two CDR2 loops interact primarily with the MHC. Given the distinct nature of class II vs. I MHC expression, peptide binding and the differential interactions with CD4 and CD8 T cell subsets, the TCR-pMHCII interaction was structurally defined. The first x-ray crystal structures of a TCR-pMHCII ternary complex are described herein. The complex contains the V module of the D10 TCR [single chain (sc) D10] derived from AKR/J (H-2^{k}) mouse T cell clone D10.G4 and a fragment of conalbumin (CA) bound to the self-I-A^{k} molecule (17, 18). A striking difference in TCR docking topology relative to TCR-pMHCI complexes is noted.

### Example 3

### Overview of the Complex Structure

The crystal structure of the scD10-CA/I-A^{k} complex was determined with molecular replacement and alternative cycles of model building and refinement. Crystals of the ternary complex were grown using the conventional hanging droplet vapor diffusion method at room temperature.

The scD10 TCR, constructed by PCR, consists of 237 residues and was organized from—to C-terminus as follows: Vβ8.2 (residues 3-110)-linker (GSADDAKKDAAKKDG)-Vα2 (AV225) (residues 1-112) with a Cys235Ser mutation. This linker (SEQ ID NO:2) was modified from that previously utilized for NMR studies (20) since the longer linker failed to give rise to I-A^{k} co-crystals of diffraction quality. For bacterial expression, the T7 promoter expression vector pET-11a was used. An overnight preculture of transformant (20 ml) was inoculated into 1L fresh Luria broth supplemented with 50 μg/ml carbenicillin at 37° C. and grown until the OD600 reached 0.6. IPTG was added to a final concentration of 1 mM and a further 4 h incubation was performed. Cells were harvested by centrifugation and lysed by sonication. The inclusion bodies were washed and dissolved in 100 mM Tris-HCl (pH 8.0) containing 6 M guanidine chloride, 10 mM EDTA and 10 mM DTT. An efficient refolding was achieved by diluting rapidly into a refolding buffer (50 mM Tris-HCl, pH 8.0, 400 mM arginine, 2 M urea, 2 mM EDTA, 4 mM reduced glutathione and 0.4 mM oxidized glutathione). The refolded material was then applied to a 3D3 affinity column followed by gel filtration on Superdex 75 and exchanged to crystallization buffer (20 mM sodium acetate, pH 5.0 with 0.025% sodium azide).

The undeglycosylated CA/I-A^{k} from CHO Lec3.2.8.1 cells were prepared as follows: a 13 residue hen egg CA peptide (residues 134-146) which is recognized by D10 TCR was fused (48) to the N-terminus of the mature I-A^{k} β chain via a flexible linker. The 37 residue leucine zipper (LZ) sequences (49) were attached to both the α and β chains, with ACID-p1 to the β chain and BASE-p1 to the α chain via flexible thrombin-cleavable linkers. The cDNA constructions were subcloned into the pEE14 vector and expressed in Lec3.2.8.1 CHO cells. The screening of secreted recombinant protein in the culture supernatant was performed by both sandwich ELISA and BIAcore using antibodies specific for I-A^{k} (10.2.16) and the LZ epitope (2H11 or 13A12). The yield was ˜0.7 mg of I-A^{k}/l supernatant. The production supernatant was applied to 2H11 affinity column and I-A^{k} protein was eluted by 50 mM citrate, 20 mM Tris, 0.5 M NaCl, 10% glycerol, pH 4.0. The eluted protein was then exchanged to 50 mM Tris-HCl, pH 8.0 and cleaved by thrombin (2u/50 μg I-A^{k}) at 4° C. for 4 h. Thrombin was then removed by passage through benzamidine Sepharose 6B beads and gel filtration on Superdex 75. Subsequently, the purified I-A^{k} was exchanged to 10 mM HEPES, pH 7.0, 0.025% sodium azide for crystallization.

The E. coli expressed scD10 (52) and undeglycosylated CA/I-A^{k} from CHO Lec3.2.8.1 cells (53), prepared as described above, were mixed at a 1:1 molar ratio to a final concentration of 23 mg/ml in 0.1M Tris-HCl buffer at pH 8.5. The protein solution was further mixed with a crystallization buffer of 8% PEG 8K/0.1M Tris pH 8.5/0.01M KCl, and then sealed against a reservoir with the same buffer. These crystals belong to the space group P2₁2₁2 with unit cell parameters a=97.6 Å, b=345.3 Å, and c=97.7 Å. There are two complexes in asymmetric unit with 78% solvent. Crystals were stepwisely transferred to cryoprotectant solution that contains 30% glycerol in addition to the crystallization buffer before freezing. One data set was collected at the SBC-CAT of Advanced Photon Source (APS) at the Argonne National Laboratory with an APS1 mosaic 3×3 CCD detector under 100° K. The wavelength used was 1.069 Å. Data were processed using programs DENZO and SCALEPACK (54). The structure was solved with molecular replacement using AMoRe (55). The refined structure of CA/I-A^{k} (56) was taken as the search model. At the beginning, only one of the CA/I-A^{k} pMHC (molecule A) was identified. The CA/I-A^{k} molecule B was located only after the first one was rigid body refined and fixed. The rigid body refinement of the two I-A^{k} molecules was then carried out, each of the Ig-like domains and the bound peptide being treated as one rigid body. A few degrees of rotations were seen for the α2 and β2 domains. After positional and individual B-factor refinement, the Rfree dropped, and the Ig-like domains of scD10, especially the one in complex-A, were already visible in the calculated 2F_{O}-F_{C} difference map. Cycles of model building and refinement gradually improved the density, allowing the correct sidechain assignment and eventually the completion of the model building and refinement. All the refinement was done using the program X-PLOR (57), and model building with program O (50). Ten percent of reflections were set aside for Rfree calculation. In the current model, each complex contains residues 1-182 and 2-190 of 1-A^{k} α and β chains, respectively, all 16 residues of the bound peptide (3 leader derived and 13 CA-derived), as well as residues 2-117 and 3-116A of D10 Vα and Vβ domains, respectively. Ten carbohydrate moieties were modeled in three potential glycosylation sites in I-A^{k} molecules. At this resolution no water molecules were included. The final 2F_{O}-F_{C} map is of excellent quality, particularly in the TCR regions and the interface between TCR and pMHC. There are very few density breaks, mainly in the BC loops of the I-A^{k}β2 domains.

TABLE 1
CRYSTALLOGRAPHIC ANALYSIS
Data collection

| Resolution limit (last shell) | 30.0-3.2 Å (3.31 Å-3.20 Å) |
|---|---|
| Reflections | |
| total number | 501,406 |
| unique | 52,592 |
| I/σ (I) | 10.0 (2.2) |
| Completeness | 95.2% (87.1%) |
| Rmerge | 7.0% (29.2%) |

Refinement statistics

| | |
|---|---|
| Resolution range | 15.0-3.20 Å |
| Number of reflection* | 46,332 (F > 0) |
| Rwork | 24.7% |
| Rfree | 29.3% |

Rms deviations

| | |
|---|---|
| Bonds | 0.007 Å |
| Angles | 1.4° |
| Dihedrals | 30.2° |
| Impropers | 0.7° |

Ramachandran plot

| | |
|---|---|
| Favored | 71.4% |
| Allowed | 21.7% |
| Generous | 6.9% |
| Unfavored | 0% |

Rmerge = Σ(|Ii(hkl)− < Ii(hkl) > |)/ΣIi(hkl)
*in working set.

In the asymmetric unit there are two complexes related to each other by a 115° rotation. In fact, the complex-A and D10-B pack together to form layers perpendicular to the longest Y axis, whereas the I-A^{k} molecules B connect layers in a fashion analogous to pillars between different floors in a building, thus leaving spaces filled with large amounts of solvent. The structures of the two complexes are very similar. The root-mean-square deviation (Rmsd) value of Cα superposition is only 0.8 Å for the whole complex. Consequently, only complex A is discussed further.

Analysis of the scD10-CA/I-A^{k} complex was performed on a model generated using the program MOLSCRIPT (56). The secondary structures, β-strands and α-helices of all component domains were defined by the program DSSP (58). CA/I-A^{k} was crystallized with the hanging droplet vapor diffusion method by equilibrating 7.2 mg/ml protein solution against a buffer of 16% isopropanol/18% PEG 4K/0.1M sodium citrate at pH 5.6 in reservoir. The crystals belong to space group P2₁2₁2, with unit cell parameters a=99.1 Å, b=122.4 Å, and c=68.4 Å. One single crystal was transferred to the cryoprotectant solution of 30% glycerol/18% PEG 4K/0.1M sodium citrate for half an hour before dipping into liquid nitrogen for freezing. A 95% complete data set was collected at Brookhaven NSLS X12C beamline using wavelength 1.072 Å with Brandeis CCD detector. The data were integrated and scaled by DENZO and SCALPACK (50). The Rmerge of the data was 9.2% to 3 Å resolution for 19,854 unique reflections. The structure was solved by molecular replacement method with AMoRe (51), using a structure of I-A^{k} complexed with a peptide from hen egg lysozyme residues 50-62 (PDB code liak) as a search model. The structure was refined with X-PLOR. The final Rfree is 29.5%, and Rwork is 23.1%.

The immediately striking observation on the structure was that the scD10 molecule sits on top of the MHC with its longer dimension crossing the bound peptide in an orthogonal manner, rather than the “diagonal” mode commonly recognized in structures of TCR-pMHCl complexes (5). The Va domain of scD10 contacts the P1 helical region of I-A^{k}, whereas the Vβ domain touches the α1 helical region. Table 2 lists all contacts between the D10 TCR and the CA/I-A^{k} pMHC ligand. In contrast to the class I pMHC-TCR ternary structures, the much longer peptide stretches out both sides of the TCR-MHC complex. In particular, the C-terminal three residues have no interaction with either TCR or MHC. The orthogonal orientation for the TCR-pMHCII interaction noted herein excludes the possibility that direct TCR contact with C-terminal peptide flanking residues is the basis for any observed functional dependence on this peptide segment in T cell recognition (19).

TABLE 2
ATOMIC CONTACTS BETWEEN THE D10 TCR
AND THE CA/I-A^{k} pMHC LIGAND

| | | | pMHC residue | |
|---|---|---|---|---|
| CDR | TCR residue | | Hydrogen bond | van der Waals contacts |
| CDR1α | Asp26 | Oδ1 | | P-1-Ser |
| | Ser27 | O | | Thr77β |
| | Thr28 | Oγ1 | | P2-Arg |
| | | Cγ2 | | P2-Arg |
| | | O | P2-Arg Nη1 | P2-Arg |
| | Phe29 | O | | Thr77β |
| | Asp30 | Cβ | | P2-Arg, Thr77β, Ala73β |
| | | Cγ | | Arg70β, Ala73β |
| | | Oδ1 | Arg70β Nη1 | Arg70β, Ala73β |
| | | Oδ2 | Arg70β Nη1, | Arg70β |
| | | | P2-Arg Nη1 | |
| | Tyr31 | Cβ | | Arg70β |
| | | Cγ | | Arg70β |
| | | Cδ1 | | Arg70β |
| CDR2α | Ser50 | Oγ | | Glu69β |
| | Leu51 | Cδ1 | | Thr77β |
| | Val52 | Cβ | | Glu69β |
| | | Cγ1 | | Arg72β, Glu69β |
| | | Cγ2 | | Glu69β, Ala73β, Arg72β |
| HV4α | Lys68 | Nζ | Asp76β Oδ2 | |
| CDR3α | Thr93 | Cγ2 | | Arg70β |
| | Gly99 | Cα | | P2-Arg, Arg70β |
| | | C | | P5-Ile |
| | | O | | P2-Arg, P5-Ile |
| | Ser100 | N | | P5-Ile |
| | | Cα | | P5-Ile |
| | | C | | P5-Ile |
| | | O | | Gln61α |
| | Phe101 | N | Arg70β Nη2 | P5-Ile |
| | | Cα | | Arg70β |
| | | Cβ | | P5-Ile |
| | | Cγ | | P5-Ile |
| | | Cδ1 | | P5-Ile, P6-Glu, P7-Trp |
| | | Cε | | P7-Trp, P8-Glu |
| | | Cζ | | P8-Glu |
| | | O | Gln61α Oε1 | |
| CDR1β | Asn31 | Oδ1 | Gln61α Oε1 | |
| CDR2β | Tyr48 | Cε2 | | Gln57α |
| | | Cζ | | Gln57α |
| | | Oη | Gln57α Oε1 | |
| | | | Gln57α Nε2 | Gln57α |
| | Tyr50 | Cδ1 | | Leu60α |
| | | Cε1 | | Leu60α, Gln57α, *Gln61α |
| | | Cδ2 | | Gln61α |
| | | Cε2 | | Gln61α |
| | | Cζ | | Gln57α, Gln61α |
| | | Oη | Gln57α Nε2 | |
| | | | Gln57α O | Gln57α, Gln61α |
| | Thr55 | C | | *Lys39α |
| | | O | Lys39α Nζ | |
| | Glu56 | Cβ | | Gln57α |
| | | Cδ | | Lys39α |
| | | Oε1 | Lys39α Nζ | Lys39α |
| | Lys57 | O | | Gln57α |
| CDR3β | Gly96 | O | P8-Glu Oε1 | |
| | Gln97 | Cα | | *Tyr67β, P8-Glu |
| | | Cβ | | Tyr67β, P8-Glu |
| | | Oε1 | Tyr67β Oη | |
| | | C | | Tyr67β |
| | Gly98 | N | Tyr67β Oη | Tyr67β |
| | | Cα | | Tyr67β, P7-Trp |
| | | C | | Tyr67β |
| | Arg99 | Cγ | | Tyr67β |
| | | Nε | Gln64β O | |
| | | Cζ | | Gln64β |
| | | Nη1 | Gln64β Oε1 | |
| | | Nη2 | Gln64β O | Gln64β |

*These residues are <50% conserved. All other MHC residues are >60% conserved in ˜100 murine β MHC class II molecules and ˜30 murine α MHC class II molecules according to Kabat tables of variability. Bonds were determined using the program CONTACTSYM (25). Given the resolution of the current structure, the atomic contacts cannot be determined with absolute precision.

An omit map in the bound peptide region showed the core of CA (P-1 to P8) to be involved in TCR-based immune recognition. A sigma A weighted 2F_{O}-F_{C} omit electron density map contoured at 1.0σ was generated using the program O (50) and prepared using a cover radius around the atoms. The omit map was generated by omitting the CA peptide entirely and after a round of torsion angle dynamics calculation.

While there have been several structures of class I-restricted α β TCRs or derivative fragments (3-5), our scD10 represents the crystal structure of a class II-restricted α β TCR V module in complex with its cognate pMHC partner. The structure of the Va-Vβ heterodimer is very similar to the recently published NMR structure of an unligated scD10 (20). Rmsd's for all of the backbone atoms of residues in β-strands between structures in the NMR ensemble and the crystal structure are 1.3 Å and 1.4 Å for Vα and Vβ domains, respectively. Notably, there does not appear to be any significant three-dimensional structural difference between TCRs that recognize peptides bound to class I vs. II MHC molecules. The human class I HLA-A2/Tax-specific B7 TCR is by far the most structurally similar to the murine class II-specific scD10 described herein. Virtually the entire V module of these two TCRs can be superimposed. The Rmsd values of the superposition for the entire Vα domain's 110 Cα atoms (excluding the first residue which is not seen in the density map of our scD10 structure) and 107 Cα atoms of the Vβ domain (excluding part of the CDR3) are only 0.98 Å and 0.72 Å, respectively. Moreover, if the two Vα domains are superimposed, then the orientations of two Vβ domains differ only by a 3.7° rotation, indicating that Vα-Vβ dimerization is very similar for these two TCRs as well.

### Example 4

### The Orthogonal Binding Mode

The orthogonal docking mode was not correctly predicted by either extensive mutagenesis studies (17) or modeling using a scD10 NMR structure in conjunction with a CA/I-A^{k} crystal structure as a starting point (20). To establish a quantitative and comparative measurement of binding orientation among TCRs and their pMHC ligands, an angle was defined between two vectors. One vector passes through the mass centers of the Vα and Vβ domains of the TCR, while the other is drawn from the N-terminal Cα atom at the P1 position (the first residue bound in the P1 pocket of the MHC) to the C-terminal Cα atom (the P9 position, the last buried residue) of the bound peptide. The angle for scD10-CA/I-A^{k} complex is 80°, very close to a right angle. Table 3 lists the orientation angle calculated for all known TCR-pMHC complex structures.

TABLE 3
THE ORIENTATION ANGLE OF A TCR ONTO A pMHC LIGAND*

| TCR-peptide/MHC Complex | Orientation angle (°) | MHC Class |
|---|---|---|
| D10-CA/I-A^{k} | 80 | II |
| 2C-dEV8/H-2K^{b} | 45 | I |
| N15-VSV8/H-2K^{b} | 54 | I |
| A6-Tax//HLA-A2 | 56 | I |
| B7-Tax/HLA-A2 | 70 | I |
| *The orientation angle of a TCR on MHC is defined as the angle between two vectors determined for the orientation of the TCR and pMHC, respectively. The vector representing the TCR direction is drawn from the mass center of Vα to the mass center of Vβ. The vector representing the pMHC complex direction is drawn from the N-terminal Cα atom to the C-terminal Cα atom of the peptide in the case of MHC | | |
| #class I. In the case of MHC class II, the vector is drawn from the P1 residue to the P9 residue of the peptide. Note that in Teng, et al. (5), twist and tilt were used for semi-quantitative comparison among different TCR-MHC complexes. Essentially, the twist and tilt angles are two projections of the orientation angle more accurately defined here. While the twist is a top view from the TCR towards the MHC, the tilt is a side view, perpendicular to the bound peptide. | | |

Note that for class I complexes, the peptide vector is defined between the anchoring residues at the two termini. The angles for TCR-pMHCI complexes span a broad range, from diagonal (45°) to close to orthogonal (70°). Differences between orthogonal and diagonal docking were seen in a comparison of the scD10-CA/I-A^{k} structure and the 2C-dEV8/H-2K^{b} (PMHCI) complex, by comparing a stereo view of scD10 (VαVβ) on CA/I-A^{k} with a stereo view of 2C (VαVβ) on dEV8/H-2K^{b}.

Garboczi et al (5) argue that in pMHCI structures, there are two high “peaks” near the N-termini of the α-helical regions forming the side wall of the peptide binding groove. These two “peaks” limit the TCR-pMHC class I binding to a diagonal mode such that the TCR can fit at a low enough point on the MHC surface to contact the entire complexed antigenic peptide. Teng et al. (5) have compared three TCR-pMHC class I complex structures, and identified a common docking mode of the TCR relative to the MHC with substantial variation of twist, tilt and shift.

Furthermore, it was noticed that the inherent left-handed twist of the eight-stranded β-sheet that forms the platform of the binding groove is the structural basis for the breaks in the two helical regions, resulting in the formation of high “peaks”. In this context, an MHC class II molecule is similar to an MHC class I molecule because all MHC molecules have the same platform. However, there are distinct features to the mode of peptide binding between the two classes. In the class I system, the 8-10 residue peptide has its termini anchored into two binding pockets whose unique chemical environments determine the polarity of the bound peptide. In addition, the bulky sidechains of the conserved Trp167α and Tyr84α from the MHC molecule occlude the peptide-binding groove at both ends. In class II MHC, these blocking sidechains are replaced by smaller ones and/or reoriented; the open ends eliminate the peptide length restriction. Moreover, the peptide (15-20 aa in length) binds to the class II MHC molecule with hydrogen bonds not just at the termini, but throughout the entire peptide via mainchain atoms (for review, see 12).

The hydrogen-bonding pattern between the CA peptide and the I-A^{k} molecule which is conserved in other pMHC class II structures was analyzed. Ten hydrogen bonds between the CA and I-A^{k} are conserved in known pMHCII structures. Compared with the class I system, the P-3 to P-1 segment is an extension. This extension plays a unique role in the orthogonal docking mode. The peptide binding groove is much wider in the middle relative to its tapered ends so that the MHC class II molecule needs to use sidechains of multiple conserved residues from α1 and β1 helical regions to reach the peptide mainchain atoms. The residues include asparagine and glutamine which form bidentate hydrogen bonds to the peptide backbone. This H-bonding pattern determines the peptide binding polarity in the class II MHC system (9-12). An important characterization of class II MHC molecules is that the α1 helix is two turns shorter in the N-terminus than the corresponding class I MHC molecule α1 helix. In particular, from Arg52α to Glu55α, the helix is replaced by an extended strand that reaches close enough to the N-terminal extension segment of the bound peptide to form a mini parallel β-sheet using mainchain atoms. The pair of mainchain-mainchain hydrogen bonds between Arg53α of the MHC class II molecule and the P-2 and P1 residues at the N-terminal part of the peptide are conserved among all known pMHCII structures. The beginning of the α1 helix, Gln57α, is at the high “peak”, so from Glu55α to Arg52α toward the N-terminus the chain runs down, away from the TCR binding surface. However, the left-handed twist of the mini β-sheet then forces the N-terminus of the peptide to point in the opposite direction, curving up toward the TCR binding surface. Together, the extended N-terminus of the bound peptide and the MHC molecule now form a broader high “peak”, or a small protruding “ridge”. A stereo view of the molecular surface of I-A^{k} (α1β1) together with the CA peptide shows the surface topology of the D10 docking platform on the CA/I-A^{k} ligand, which can be compared with the same view of pMHCI taken from the 2C-dEV8/H-2K^{b} structure, demonstrating the stereo view of the molecular surface of H-2K^{b} (α1/α2) together with the dEV8 peptide (5) and showing the smaller high point on the left side of the docking platform for the MHC class I-restricted TCR molecule. The peptide is mostly buried and makes little, if any, contribution to the elevated points.

The scD10-CA/I-A^{k} structure shows that a diagonal TCR docking would result in a collision between the Vα domain of TCR and the pMHCII on the “left” side. Moreover, the tilt angle of a TCR relative to an MHC molecule (see Table 3 legend for the definition of tilt angle) exacerbates this potential clash by maintaining the Vα domain in close proximity to MHC. It is proposed that while the TCR-pMHC class I docking may have more variation in terms of the orientation angle as demonstrated in Table 3, the topology of TCR binding to pMHC class II may be more closely restricted to an orthogonal mode due to the “ridge” described above. It is interesting to note that the protrusion of the peptide's N-terminus has been suggested as a site for disruption by DM in the process of exchanging CLIP for an antigenic peptide in the MHC class II molecule (21).

### Example 5

### The Interface

The interaction between D10 and CA/I-A^{k} buries 1718 Å² of surface area, 861 Å² from the pMHC and 857 Å² from the TCR using a 1.7 Å probe (22). Twenty-three percent of the pMHC buried surface involves the peptide. In general, the size of the buried surface is comparable to that previously reported for three class I ternary structures (1700-1880 Å²). However, the Sc value (the shape correlation statistic, a measurement of the degree of geometric match between two juxtaposed surfaces, where interfaces with Sc=1 fit perfectly whereas interfaces with Sc=0 effectively define topologically uncorrelated surfaces (see Lawrence & Coleman, ref. 22) of the interface between the scD10 VαVβ module and the CA/I-A^{k} ligand is 0.70, higher than for class I TCR-pMHC interfaces whose Sc values range from 0.45 to 0.64 (23, 24). Moreover, the number of atomic contacts (25) in our class II complex structure is about twice as many as those for the class I complexes. For I-A^{k}, 68 atomic contacts exist with D10. By contrast, there are just 27H-2K^{b} contacts with the 2C TCR, and 27 and 34 HLA-A2 contacts for the A6 and B7 TCRs, respectively. These results suggest a much better shape complementarity of the scD10-CA/I-A^{k} interface, and agree well with the higher affinity of D10 for its pMHC ligand relative to that of 2C, for example (1-2 μM vs. 100 μM) (5, 26). Of particular relevance is the finding that the additional interface atomic contacts can largely be ascribed to contacts between the TCR and the I-A^{k} molecule rather than between the TCR and the CA peptide (Table 2). Assuming that these results are representative for other class II MHC-specific TCRs, the dominance of this TCR-MHC class II contact may explain why expression of a single pMHC class II complex in the thymus can select many different TCRs (27). The data also show that despite having roughly the same buried surface area, the complementarity of TCR-pMHC recognition surfaces can vary substantially from a low extreme to one even better than that of antigen-Fab complex, as is the case for the scD10-CA/I-A^{k} complex. In complexes like 2C-dEV8/H-2K^{b}, a few large cavities (5) contribute to poor shape complementarity. The presence or absence of such cavities may vary for different TCR-pMHC complexes, thereby influencing the shape of complementarity.

Of the total buried surface area, Vα accounts for 519A² while VP accounts for 338 Å² of the TCR buried surfaces. This result is consistent with the notion that Vα dominates in the interaction which is generally true for the class I system as well. The calculations showed that the buried surface areas of Vα and Vβ are 480 Å² and 430 Å² for the 2C-dEV8/H-2Kb complex, 576 Å² and 319 Å² for A6-Tax/HLA-A2, and 555 Å² and 260 Å² for B7-Tax/HLA-A2, respectively. Perhaps more importantly, amongst different TCR-pMHC complexes, the variation in buried surfaces is significantly smaller for Vα than for Vβ. Given that the rotation angle of known TCRs relative to their MHC ligands varies as much as 35° (Table 3), these data suggest that the pivot point is closer to Vα so that the Vα domain location on the pMHC will not change as much as the Vβ domain which can alter dramatically. Differences in the disposition of CDR loops reflect this pivot point; these differences were seen in models prepared using the program GRASP (51).

Variability in TCR docking also arises from differences in the tilt angle as described by Teng et al. (5) and noted in the Table 3 legend. The extreme is the A6-Tax/HLA-A2 structure, where the large tilt essentially precludes CDR1b and CDR2b from making contact with the MHC molecule (5). Given that Vα is critical for TCR selection in thymic development as well as mature T cell activation (28), this Vα dominance in immune recognition is not unexpected.

Comparison between the scD10 TCR interaction with CA/I-A^{k} analyzed herein and the 2C TCR interaction with dEV8/K^{b} (5) shows how a single TCR Vβ8.2 domain can bind in distinct orientations to class I and class II pMHC ligands. In the 2C-dEV8/Kb complex, the germline Vp8.2 segment recognizes the K^{b} α1 helical MHC residues Gln72, Val76 and Arg79 via CDR2, and the K^{b} α2 helical residues Lys146, Gln149 and Ala150 via CDR1 (5). In the scD10-CA/I-A^{k} complex, the identical germline Vβ8.2 segment recognizes the I-A^{k} α1 helical residues Lys39, Gln57 and Leu60 via CDR2, and Gln6l via CDR1. Given that these two docking interactions are to highly conserved MHC class I and to highly conserved class II residues, respectively, it appears that the Vβ domain plays a major role in MHC recognition by both classes of TCRs and perhaps in pre-TCRs as well (29).

### Example 6

### Antigenic Peptide Recognition

Although the peptide in the ternary structure is 16 residues in length, designated as from P-3 to P13, the TCR interaction is restricted to the P-1 to P8 segment. Table 2 lists all the contacts to the peptide. It is noteworthy that of 27 atomic contacts with the peptide, 23 involve Vα and only four involve Vβ. This dominance of the Vα domain in peptide recognition was not appreciated previously, although early molecular modeling efforts correctly suggested that an orthogonal TCR docking mode was possible (Davis & Bjorkman, ref. 2). The spiral conformation of bound peptide (12) dictates that of the deeply buried peptide residues, only those at positions P2, P5 and P8 are accessible to the TCR molecule. The Trp at the P7 position is an exception due to its bulky indole ring, which is partially exposed on the TCR binding surface. As for the rest of the peptide, the backbone of the P-2 residue is engaged in a mini parallel β-sheet with the MHC molecule as discussed above, while the P-3 and the C-terminal three residues (P11-P13) have no contacts with either MHC or TCR whatsoever, though well defined by unambiguous densities.

The P2 residue is an Arg. It forms multiple salt bridges with both Asp30α in the D10CDR1α and I-A^{k} Glu74β, respectively. The same TCR Asp30α also interacts with I-A^{k} Arg70β. Moreover, the upward-pointing P2-Arg is within van der Waals contacts to backbone of CDR3α Gly99α and CDR1α Thr28α (see Table 2). This knitted local structure packs closely onto the sidechain of Ile at the P5 position from the N-terminal side of the peptide. The P5 residue is important structurally and biologically. Alteration of this residue adversely affects D10-TCR recognition of CA/I-A^{k} (17). The sidechain of Ile at P5 fits extremely well into a hydrophobic pocket. Apart from the neutralized network discussed above, on the C-terminal peptide side is the indole ring of the Trp at P7 stacking onto the isobutyl group of the P5-Ile. On the top, from the TCR direction, the P5-Ile contacts the backbone of the tip of CDR3α which consists of Gly99α-Ser100α-Phe101α. The phenolic ring of Phe101α bends towards the P7-Trp position. The exposed tip of the indole ring of P7-Trp makes contacts with the Phe101α aromatic ring. The other peptide residue engaged in recognition is the P8-Glu residue. P8-Glu forms bifurcated hydrogen bonds to sidechains of Tyr60β and Tyr67β of the I-A^{k} molecule. Only the aliphatic portion of the P8-Glu sidechain makes van der Waals interactions with CDR3α Phe101α and the aliphatic part of CDR3β Gln97β. In addition, there is one H-bond between the carbonyl oxygen of Gly96β and the sidechain of P8-Glu. Together, it appears that the TCR recognition of the particular antigenic peptide in question is largely hydrophobic and involves a number of backbone associations with the TCR molecule. Although scD 10 TCR recognition of CA/I-A^{k} is centered at the P5 position, it is also coordinated with interactions to peptide residues at P2, P7 and P8. The observed contacts are consistent with studies mapping the D10 footprint onto CA/I-A^{k} (18). For example, immunization of D10 TCR Vα2 tg mice with the Glu8φAlαCA peptide variant gives rise to T cell hybridomas, all of which use VP8.2 but with variation in CDR3β, consistent with the view that CDR3p is involved in recognition of the P8 position. Immunization of D10 TCR α tg or D10 TCR β tg with the IleφLys CA variant both failed to generate specific hybridomas, implying that CDR3α and/or β may be important for Ile5Pro recognition. Immunization of D10TCR β tg mice with the Arg2φAsp CA variant resulted in a switch in Vα usage from Vα2 to Vα8, suggesting that the germline CDR1 and/or 2 loops interact with this peptide residue.

### Example 7

### Implications for Class II MHC-Based Immune T Cell Recognition

The current sc D10-CA/I-A^{k} complex offers several insights into immune recognition of other pMHC class II ligands by other TCRs. First, the size of a TCR footprint on the MHC covers maximally nine peptide residues (˜25 Å). Hence, while MHC class II molecules capture peptides of substantially larger length, only a subset of residues is “read out” by the bound TCR. Second, the P5 residue of the MHC-bound peptide occupies the central position [corresponding to the P4 position of the MHC class I-bound peptide (28)]. As such, this central, solvent exposed residue is critically important for the TCR binding process. Therefore, even a minor conservative substitution at this residue can destroy binding (i.e. null ligand) or lead to altered peptide ligands with very weak agonist or in fact, antagonist activity (31-33). Third, for all class II molecules examined, there appear to be 3-4 pMHC binding pockets (at P1, P4, P6 and P9 for I-A^{k}, I-E^{k} and DR and P1, P4 and P9 for I-A^{d}). Only several upward pointing peptide residues can serve as direct TCR contacts.

Based on the observed molecular envelope of the TCR and the observed orthogonal orientation for class II MHC-restricted TCR interaction, it appears that these basic principles apply in a general way to recognition of multiple pMHCII ligands including HEL₄₈₋₆₂/I-A^{k} (10, 34), Hb₆₄₋₇₆/I-E^{k} (35), moth cytochrome C (MCC)₉₃₋₁₀₃/I-E^{k} (31) and DR2-restricted myelin basic protein (MBP)₈₅₋₉₉ (36, 37). Moreover, it has been suggested that a single TCR can recognize multiple pMHCII ligands (36, 37). As the class II specific TCR focuses on the central P5 residue, mutations which affect non-P5 positions may be less detrimental to the recognition process.

Despite overall structural similarity, CDR3 conformations appear to differ between free and complexed scD10. In the x-ray structure of the complex, the CDR3 loops are close to one another. Packing among the sidechain of Gln106β, Ala104β and Leu104β forms a hydrophobic core between the CDR3 loops. In contrast, there is no evidence that CDR3α packs with CDR3β in unligated scD10. Numerous NOEs are observed between the methyl group of Ala104β and other CDR3β residues (Gly96, Gln197, Arg99, Glu105), but contacts to Leu104α or other CDR3α residues are not observed. In all of the calculated NMR structures, the CDR3α and CDR3β loops are well separated. The backbones of both CDR3s are also highly mobile on the picosecond timescale in the free protein (20), suggesting that they are not tightly packed. Hence, during immune recognition, the mobile CDR3 loops of scD10 assume their pMHCII binding conformation, clamping down on the central peptide region.

### Example 8

### Structural Basis of Alloreactivity

Approximately 1-10% of peripheral T cells are able to recognize allogeneic MHC molecules to which they were never exposed (38). The precise molecular basis of alloreactivity is yet to be fully defined. In this regard, the complex of scD10-CA/I-A^{k} is informative since the D10 TCR not only recognizes the antigenic CA peptide bound to I-A^{k} but also responds to all MHC class II molecules whose I-A β chain contains the sequence “PEI” at positions 65-67, including I-A^{b, v, p, q, d} (17). In comparison, MHC class II molecules having a Tyr at this position such as I-A^{f, k, r, s, u, g7}, cannot stimulate D10 cells in the absence of the CA peptide. Various mutagenesis studies conducted on D10 showed that a hybrid I-A^{k}α/I-A^{b}β MHCII molecule can stimulate D10 cells in the absence of exogenous antigen, suggesting that polymorphic residues critical for alloreactivity are located on the I-A β chain.

In order to elucidate this source of alloreactivity from the structural perspective, the CA/I-A^{k} ligand and the alloreactive I-A^{d} molecule were compared. The latter was taken from the recently solved x-ray structure of ovalbumin (323-339) complexed with I-A^{d} (11). Since all residues from I-A^{k} involved in the interaction with D10 as listed in Table 2 are conserved in I-A^{d} with the exception of residues Tyr67 in I-A^{k} and Pro65-Glu66-Ile67 (PEI) in I-A^{d}, it is likely that D10 docks onto I-A^{d} in the same way as onto I-A^{k}. A model using the I-A^{d} superimposed onto the scD10-CA/I-A^{k} complex was assessed for differences.

The major structural difference involves the β chain residues Pro65, Glu66 and Ile67 in I-A^{d} which form a protrusion interrupting the β chain α helix. As a consequence, in I-A^{d}, residue Ile67 assumes a similar position to residue Tyr67 in I-A^{k}. The sidechains and mainchain atoms on the CDR1 and 2 loops of D10 Vα make no hydrogen bonds or salt bridges to I-A^{k} β1 helix residues. A model of scD10 bound to I-A^{d} was also assessed, based upon superposition of I-A^{d} and I-A^{k}. The α1 H2 helix and the β 1 region between and including H2α-H2β of the two class II MHC molecules were superimposed (46 Cα's, Rmsd=0.55 Å). The potential interactions between Glu66 from the PEI₆₅₋₆₇ motif of I-A^{d} to Tyr31 of CDR1 and Ala48, Ser50 and Lys56 of CDR2 of D10 Vα were examined.

The aliphatic sidechain of the Ile67 in I-A^{d} can replace the aromatic ring on the sidechain of Tyr67 in I-A^{k}, forming van der Waal's contacts with the VP CDR3 loop. To avoid steric clashes, side chains from residue Arg99 of D10 VP and residue Glu66 of 1-A^{d}β1 are rotated and the mainchain conformation around PEI on I-A^{d} is slightly modified. The backbone NH vectors of residues directly adjacent to Arg99 are among the most mobile in scD10 (20).

Glu66 can form multiple potential interactions with CDR1 and CDR2 of D 10 Vα. Additionally, the hydrogen bond between Gln64 and Arg99 from CDR3 of Vβ is preserved. Therefore, despite loss of one hydrogen bond of Tyr67 to D10 Vβ Gly98, these potential additional contacts between the CDR loops of D10 Vα and the inserted PEI residues can enhance the affinity between MHC and D10. Other TCR allo-pMHCII interactions cannot be excluded. Consistent with this view, it has also been suggested that in the case of the 2C allo-MHCI response (L^{d}), allorecognition results from increased interaction between the 2C TCR Vβ domain and the allostimulus (39). The ability of exposed MHC helical polymorphic residues to permute the number and nature of contacts with the TCR is a feature of other class II MHC-restricted allogeneic responses (40, 41). For example, the naturally occurring I-A^{b} mutant H-2^{bm12} generates a strong alloresponse in H-2^{b} mice. This molecule differs from I-A^{b} at only three position: 67β, 70β and 71β.

While self-peptides bound to MHC have been shown to play a critical role in alloreactivity against MHC class I molecules (rev. in 41), less is known about the nature of peptide ligands in class II MHC-based alloreactivity (42). Given the additional contacts between the D10 TCR and I-A^{d}, it appears that there are fewer interactions required between the peptide(s) associated with I-A^{d} molecules and the D10 TCR. However, a peptide(s) must also be involved since replacement of the PEI sequence in lieu of Tyr67 in the I-A^{k} β chain is not sufficient to create an allostimulatory molecule for the D10 T cell clone (17).

### Example 9

### Superantigen Binding

Superantigens (SAG) are a family of immunostimulatory and disease-causing proteins derived from bacterial or endogenous retroviral genes which are capable of activating a large fraction of the T cell population (43). In general, the activation appears to require a bridging interaction between the Vβ domain of the TCR and an MHC class II molecule. Although crystal structures (44) showing the detailed interactions between SEB, a representative bacterial SAG, and a TCR Vβ8.2 chain or SEB and the HLA-DR1 class II MHC molecule have been determined, the physiologically relevant tripartite TCR-SAG-pMHC complex has not yet been characterized. A structural model of TCR-SAG-pMHC complex was previously generated (44) based on least squares superposition of 1) the 14.3.d VβCβ-SEB complex, 2) the SEB-HLA-DR1 complex and 3) the 2C TCR α β heterodimer. However, since the docking mode of TCR on the class II MHC was structurally unknown and presumed to be similar to the observed diagonal mode of TCR on class I MHC, it was noted that the rotational orientation of the TCR and MHC molecules in the predicted TCR-SEB-pMHC complex was substantially different (˜40°) from the 2C-dEV8/Kb complex. The structural determination of the D10-CA/I-A^{k} complex herein enables us to offer additional insight into the nature of the SAG binding to TCR and pMHC.

A study of the 14.3.d Vβ8.2 Cb-SEB complex superimposed onto the D10-CA/I-A^{k} complex included least-square fitting of the Vβ domains from each complex (92 Ca's from residues Val3-Gly94 of Vβ, Rmsd=0.67 Å). Because the TCR docks on the MHC molecule in a nearly perpendicular manner, the SEB directly interacts with the MHC α1 helix without any requirement for TCR rotation. However, certain segments of SEB and the α1 helix collide. Since there is no significant conformational change observed for either of the component domains involved in this interaction, it was reasoned that a relative domain movement could alleviate any steric clash. To test this idea, I-A^{k} was removed from the complex and then superimposed the SEB HLA-DR1 complex onto the 14.3.d VβCβ-SEB complex and scD10 (VαVβ) module. The two SEB superantigen molecules were used for least-square fitting (83 Cα's, Rmsd=0.63 Å). From this second model, it was observed that the direct interaction between Vβ and the MHC α1 helix is disrupted by SAG. The key interaction site for SEB involves CDR2 (Tyr50, Ala52, Gly53, Ser54, Thr55) and certain other residues (Glu56, Lys57, Tyr65, Lys66, Ala67) as reported by Li et al. (44). In this way, the superantigen wedges itself between Vβ and the MHC class II α1 helix, forcing the MHC to swing away from V β and toward Vα while preserving the direct interaction between the Vα domain of the TCR and MHC class II β 1 helix. This latter interaction has been proposed to be critical in stabilizing the TCR-SAG-pMHC complex. In fact, T cell activation by SAG is believed to be dependent upon the interaction between a given TCR Vα domain and the MHC class II β1 helix (45). When the HLA-DR was replaced with the I-A^{k} molecule based on the structural alignment of residues of the two helices of each MHC molecule (43 Cα's, Rmsd=1.02 Å), it was estimated that the relative swing angle between TCR and MHC in the TCR-SAG-pMHCII complex compared to the TCR-pMHCII complex is ˜17°.

### Example 10

### Differential TCR Binding and Co-Receptor Selection in the Thymus

Given that there are no intrinsic structural differences between class I vs. class II MHC restricted TCR V modules as shown above, it is questioned what directs expression of a TCR to the proper CD4 or CD8 subset. During thymocyte development, progenitor cells transit from a CD4⁻CD8⁻ double negative (DN) stage through a CD4⁺CD8⁺ double positive (DP) stage and then into a CD4⁺CD8⁻ or CD4⁻CD8⁺ single positive (SP) stage (46). Selection for maturation occurs upon the interaction of thymocytes with stromal cells expressing self-pMHCI or self-pMHCII ligands within the thymus, beginning at the DP stage where the TCR first appears. Differentiation to the SP thymocyte stage, however, requires a match between the MHC class specificity of the TCR which a thymocyte bears and the CD4 or CD8 co-receptor it expresses. To explain how a thymocyte precisely coordinates co-receptor expression and TCR specificity, two models have been proposed (47). The “instruction model” argues that co-engagement of TCR and CD4 or CD8 on a DP thymocyte specifically signals the cell to move down one pathway while extinguishing the expression of the inappropriate co-receptor. On the other hand, the “selection model” postulates that cells initiate stochastically or otherwise a process which terminates expression of one of the two co-receptors. If the correct match was chosen, then the cell further differentiates but if not, differentiation is stalled.

Distinctions between class I vs. II pMHC complexes and variation in TCR docking observed herein offers strong support for the “instruction model”. It is suggested that depending on the degree of complementarity of a given TCR recognition surface and a self-pMHCI or self-pMHCII complex, binding occurs, and a diagonal docking mode with substantial variability onto pMHCI or a preferred orthogonal docking mode onto pMHCII is established. Subsequently, CD8 preferentially co-engages with the former and CD4 with the latter. Expression of the irrelevant co-receptor is then extinguished. Based on CD8-MHC class I crystal structures and on mapping of MHC class II residues involved in CD4 binding, the two co-receptors likely occupy an “homologous” orientation relative to the TCR (15, 16). Thus, it appears that the differential TCR docking to self-pMHCI vs. self-pMHCII contributes specificity for coordination of appropriate co-receptor selection.

### REFERENCES

1. E. L. Reinherz, S. C. Meuer, S. F. Schlossman, Immunol Rev 74, 83-112 (1983); P. Marrack, J. Kappler, Adv Immunol 38, 1-30 (1986); H. Clevers, B. Alarcon, T. Wileman, C. Terhorst, Ann Rev Immunol 6, 629-662 (1988); J. D. Ashwell, R. D. Klausner, Ann Rev Immunol 8, 139-167 (1990).

2. O. Acuto, et al., Proc Natl Acad Sci USA 81, 3851-3855 (1984); M. Fabbi, O. Acuto, J. E. Smart, E. L. Reinherz, Nature 312, 269-271 (1984); G. K. Sim, et al., Nature 312, 771-775 (1984); Y. Chien, et al., Nature 312, 31-35 (1984); N. R. Gascoigne, Y. Chien, D. M. Becker, J. Kavaler, M. M. Davis, Nature 310, 387-391 (1984); S. M. Hedrick, E. A. Nielsen, J. Kavaler, D. I. Cohen, M. M. Davis, Nature 308, 153-158 (1984); S. M. Hedrick, D. I. Cohen, E. A. Nielsen, M. M. Davis, Nature 308, 149-153 (1984); P. Patten, et al., Nature 312, 40-46 (1984); H. Saito, et al., Nature 312, 36-40 (1984); Y. Yanagi, et al., Nature 308, 145-149 (1984); A. C. Hayday, et al., Nature 316, 828-832 (1985); J. Novotny, S. Tonegawa, H. Saito, D. M. Kranz, H. N. Eisen, Proc Natl Acad Sci USA 83, 742-746 (1986); C. Chothia, D. R. Boswell, A. M. Lesk, EMBO J 7, 3745-3755 (1988); M. M. Davis, P. J. Bjorktnan, Nature 334, 395-402 (1988).

3. G. A. Bentley, G. Boulot, K. Karjalainen, R. A. Mariuzza, Science 267, 1984-1987 (1995); B. A. Fields, et al., Science 270, 1821-1824 (1995); D. Housset, et al., EMBO J. 16, 4205-4216 (1997).

4. K. C. Garcia, et al., Science 274, 209-219 (1996); J. Wang, et al., EMBO J. 17, 10-26 (1998).

5. D. N. Garboczi, et al., Nature 384, 134-141 (1996); K. C. Garcia, et al., Science 279, 1166-1172 (1998); M.-K. Teng, et al., Curr Biol 8, 409-412 (1998); Y. H. Ding, et al., Immunity 8, 403-411 (1998); Y. H. Ding, B. M. Baker, D. N. Garboczi, W. E. Biddison, D. C. Wiley, Immunity 11, 45-56 (1999).

6. B. Benacerraf, Harvey Lect 67, 109-141 (1973); B. Benacerraf, J Immunol 120, 1809-1812 (1978); M. J. Bevan, Nature 269, 417-418 (1977); R. M. Zinkemagel, P. C. Doherty, Nature 248, 701-702 (1974); R. M. Zinkemagel, et al., J Exp Med 147, 882-896 (1978); R. N. Germain, Cell 76,287-299 (1994).

7. F. Momburg, J. Roelse, G. J. Hämmerling, J. J. Neefjes, J Exp Med 179, 1613-1623 (1994); M. J. Androlewicz, P. Cresswell, Immunity 1-14 (1994).

8. A. Rudensky, P. Preston-Hurlburt, S. C. Hong, A. Barlow, C. A. J. Janeway, Nature 353, 622-627 (1991); R. M. Chicz, et al., Nature 358, 764-768 (1992); D. F. Hunt, et al., Science 256, 1817-1820 (1992).

9. D. H. Fremont, M. Matsumura, E. A. Stura, P. A. Peterson, I. A. Wilson, Science 257, 919-927 (1992); D. R. Madden, J. C. Gorga, J. L. Strominger, D. C. Wiley, Cell 70, 1035-1048 (1992); M. Matsumura, D. H. Fremont, P. A. Peterson, I. A. Wilson, Science 257, 927-934 (1992); J. H. Brown, et al., Nature 364, 33-39 (1993); L. J. Stem, et al., Nature 368, 215-221 (1994); D. H. Fremont, W. A. Hendrickson, P. Marrack, J. W. Kappler, Science 272, 1001-1004 (1996); A. Dessan, C. M. Lawrence, S. Cupo, D. M. Zaller, D. C. Wiley, Immunity 7, 473-481 (1997); V. L. Murthy, L. J. Stem, Structure 5, 1385-1396 (1997).

10. D. H. Fremont, D. Monnaie, C. A. Nelson, W. A. Hendrickson, E. R. Unanue, Immunity 8, 305-317 (1998).

11. C. A. Scott, P. A. Peterson, L. Teyton, I. A. Wilson, Immunity 8, 319-329 (1998).

12. L. J. Stem, D. C. Wiley, Structure 2, 245-251 (1994); D. R. Madden, Annu Rev Immunol 13, 587-622 (1995).

13. E. L. Reinherz, S. F. Schlossman, Cell 19, 821-827 (1980).

14. S. Meuer, S. F. Schlossman, E. L. Reinherz, Proc Natl Acad Sci USA 79, 4395-4399 (1982); W. E. Biddison, P. E. Rao, M. A. Talle, G. Goldstein, S. Shaw, J Exp Med 156, 1065-1076 (1982); A. M. Krensky, C. S. Reiss, J. W. Mier, J. L. Strominger, S. J. Burakoff, Proc Nat Acad Sci USA 79, 2365-2369 (1982).

15. C. A. Doyle, J. L. Strominger, Nature 330, 256-259 (1987); R. Konig, L. Y. Huang, R. Germain, Nature 356, 796-798 (1992); U. Moebius, P. Pallai, S. C. Harrison, E. L. Reinherz, Proc Natl Acad Sci USA 90, 8259-8263 (1993); J. M. Connolly, T. H. Hansen, A. L. Ingold, T. A. Potter, Proc Natl Acad Sci USA 87, 2137-2141 (1990); R. D. Salter, et al., Nature 345, 41-46 (1990).

16. G. F. Gao, et al., Nature 387, 630-634 (1997); P. Kern, et al., Immunity 9, 519-530 (1998).

17. J. Kaye, S. Porcelli, J. Tite, B. Jones, C. A. J. Janeway, J Exp Med 158, 836-856 (1983); P. Portoles, J. M. Rojo, C. A. J. Janeway, J Mol Cell Immunol 4, 129-137 (1989); S. C. Hong, et al., Cell 69, 999-1009 (1992); S.-C. Hong, et al., J Immunol 159, 4395-4402 (1997).

18. D. B. Sant'Angelo, et al., Immunity 4, 367-376 (1996). 19. R. T. Carson, K. M. Vignali, D. L. Woodland, D. A. Vignali, Immunity 7, 387-399 (1997).

20. B. J. Hare, et al., Nat Struct Biol 6, 574-581 (1999). 21. L. Mosyak, D. M. Zaller, D. C. Wiley, Immunity 9, 377-383 (1998).

22. C. Chothia, J. Janin, Nature 256:705-708 (1975); M. L. Connolly, J Appl Crystallgr 16, 548-558 (1983); M. C. Lawrence, P. M. Coleman, J Molec Biol 234, 946-950 (1993).

23. X. Ysern, H. Li, R. A. Mariuza, Nat Struct Biol 5, 412-414 (1998).

24. K. C. Garcia, L. Teyton, I. A. Wilson, Annu Rev Immunol 17, 369-397 (1999).

25. S. Sheriff, W. A. Hendrickson, J. L. Smith, J Molec Biol 197, 273-296 (1987).

26. Our unpublished results.

27. CP. Liu, D. Parker, J. Kappler, P. Marrack, J Exp Med 186, 1441-1450 (1997); L. Ignatowicz, et al., Immunity 7, 179-186 (1997); S. Tourne, et al., Immunity 7, 187-195 (1997); C. D. Surh, D. S. Lee, W. Fung Leung, L. Karlsson, J. Sprent, Immunity 7, 209-219 (1997).

28. Y. Ghendler, et al., Proc Natl Acad Sci USA 95, 10061-10066 (1998); B. T. Backström, U. Muller, B Hausmann, E. Palmer, Science 281, 835-838 (1998).

29. Y. Shinkai, et al., Science 259, 822-825 (1993); H. J. Fehling, A. Krotkova, C. Ruf-Saint, H. von Boehmer, Nature 375, 795-798 (1995); C. N. Levelt, B. Wang, A. Ehrfeld, C. Terhorst, K. Eichmann, Eur J Immunol 25, 1257-1261 (1995); Y. Xu, L. Davidson, F. W. Alt, D. Baltimore, Proc Natl Acad Sci USA 93, 2169-2173 (1996); S. Koyasu, et al. Int Immunol 9, 1475-1480 (1997).

30. Results from specificity and sequence analysis of T cell hybridomas derived from mice carrying a given V2 or Vβ8.2 D10 TCR transgene immunized with altered peptide ligands of CA (18) are consistent with the structural information reported here.

31. P. A. Reay, R. M. Kantor, M. M. Davis, J Immunol 152, 3946-3957 (1994).

32. G. J. Kersh, P. M. Allen, J Exp Med 184, 1259-1268 (1996).

33. K. Matsui, J. J. Boniface, P. Steffner, P. A. Reay, M. M. Davis, Proc Natl Acad Sci USA 91, 12862-12866 (1994); D. S. Lyons, et al., Immunity 5, 53-61 (1996); G. J. Kersh, E. N. Kersh, D. H. Fremont, P. M. Allen, Immunity 9, 817-826 (1998).

34. P. M. Allen, et al., Nature 327, 713-715 (1987).

35. B. D. Evavold, J. Sloan-Lancaster, K. J. Wilson, J. B. Rothbard, P. M. Allen, Immunity 2, 655-663 (1995).

36. K. J. Smith, J. Pyrdol, L. Gauthier, D. C. Wiley, K. W. Wucherpfennig, J Exp Med 188, 1511-1520 (1998).

37. K. W. Wucherpfennig, J. L. Strominger, Cell 80, 695-705 (1995).

38. L. A. Sherman, S. Chattopadhyay, Annu Rev Immunol 11, 385-402 (1993).

39. J. A. Speir, et al., Immunity 8, 553-562 (1998).

40. I. F. McKenzie, et al., J Exp Med 150, 1323-1328 (1979); J. Bill, et al., J Exp Med 169, 115-133 (1989).

41. J. A. Frelinger, M. McMillan, Immunol Rev 154, 45-58 (1996).

42. C. Daniel, S. Horvath, P. M. Allen, Immunity 8, 543-552 (1998).

43. M. T. Scherer, L. Ignatowicz, G. M. Winslow, J. W. Kappler, P. Marrack, Annu Rev Immunol 9, 101-128 (1993); S. R. Webb, N. R. J. Gascoigne, Curr Opin Immunol 6, 467-475 (1994).

44. H. Li, et al., Immunity 9, 807-816 (1998); T. S. Jardetzky, et al., Nature 368, 711-718 (1994).

45. D. L. Woodland, M. A. Blackman, Immunol Today 14, 208-212 (1993); K. Daly, et al., J Immunol 155, 27-34 (1995); M. A. Blackman, D. L. Woodland, Immunol Res 15, 98-113 (1996); D. Donson, et al., J Immunol 158, 5229-5236 (1997); A. M. Deckhut, Y. Chien, M. A. Blackman, D. L. Woodland, J Exp Med 180, 1931-1935 (1994); N. Labrecque, J. Thibodeau, W. Mourad, R. P. Sekaly, J Exp Med 180, 1921-1929 (1994).

46. E. L. Reinherz, P. C. Kung, G. Goldstein, R. H. Levey, S. F. Schlossman, Proc Natl Acad Sci USA 77, 1588-1592 (1980); B. J. Fowlkes, D. M. Pardoll, Adv Immunol 44, 207-264 (1989); A. Kruisbeek, Curr Opin Immunol 5, 227-234 (1993).

47. S. H. Chan, D. Cosgrove, C. Waltzinger, C. Benoist, D. Mathis, Cell 73, 225-236 (1993); S. H. Chan, C. Benoist, D. Mathis, Immunol Rev 135, 119-131 (1993); C. B. Davis, N. Kileen, M. E. Casey Crooks, D. Raulet, D. R. Littman, Cell 73, 237-247 (1993); H. S. Teh, et al., Nature 335, 229-233 (1988); J. P. M. van Meerwijk, R. N. Germain, Science 261, 911-915 (1993); J. P. M. van Meerwijk, E. M. O'Connel, R. N. Germain, Semin Immunol 6, 231-239 (1994).

48. H. Kozono, J. White, J. Clements, P. Marrack, J. W. Kappler, Nature 369, 151-154 (1994).

49. H.-C. Chang, et al., Proc Natl Acad Sci USA 91, 11408-11412 (1994); J. Liu, et al., J Biol Chem 271, 33639-33646 (1996).

50. T. A. Jones, J-Y. Zou, S. W. Cowtan, M. Kjeldgaard, Acta Crystallogr A47, 110-119 (1991).

51. A. Nicholls, K. A. Sharp, B. Honig, Proteins: Struct. Funct. Genet. 11, 281-296 (1991).

52. Production and purification of scD10 TCR.

53. Production and purification of MHC class II I-A^{k}.

54. Z. Ortwinowski, W. Minor, Meth Enzymol 276, 307-326 (1997).

54. J. Navaza, Acta Crystallogr A50, 157-163 (1994).

56. CA/I-A^{k} was crystallized with the hanging-droplet vapor diffusion method.

57. A. T. Brünger, Yale Univ Press, New Haven, Conn. (1992).

58. W. Kabsch, C. Sander, Biopolymers 22, 2577-2637 (1983).

59. P. J. Kraulis, J Appl Crystallogr 24, 946-950 (1991).

While this invention has been particularly shown and described with references to preferred embodiments thereof, it will be understood by those skilled in the art that various changes in form and details may be made therein without departing from the scope of the invention encompassed by the appended claims.

While this invention has been particularly shown and described with references to preferred embodiments thereof, it will be understood by those skilled in the art that various changes in form and details may be made therein without departing from the scope of the invention encompassed by the appended claims.

What is claimed is:

1. A polypeptide capable of expanding thymocytes bearing a T cell receptors which are capable of recognizing disease or foreign antigen.

2. A vaccine composition comprising the polypeptide of claim 1 in a physiologically acceptable vehicle.

3. A polypeptide capable of reducing or eliminating T cell receptors on thymic cells that recognize a self antigen.

4. A vaccine composition comprising the polypeptide of claim 3 in a physiologically acceptable vehicle.

5. A synthetic thymus comprising stromal elements bearing MHC molecules and polypeptide capable of a negative or positive selection of thymocytes with T cell receptors of targeted specificity.

6. A method of influencing selection of thymocytes having T cell receptor specificity in a host, comprising administering to the host a polypeptide of interest, wherein the polypeptide of interest causes selection of thymocytes having a T cell receptor specificity in the thymus.

7. The method of claim 6, wherein the polypeptide of interest causes positive selection of thymocytes having a T cell receptor specificity.

8. The method of claim 6, wherein the polypeptide of interest causes negative selection of thymocytes having a T cell receptor specificity.

9. A method of performing thymic vaccination of a host, comprising administering to the host a polypeptide of interest, wherein the polypeptide of interest causes selection of thymocytes having a T cell receptor specificity in the thymus.

10. The method of claim 9, wherein the polypeptide of interest causes positive selection of thymocytes with a T cell receptor specificity.

11. The method of claim 10, wherein the positive selection of a T cell receptor specificity generates thymocytes with T cell receptors which are capable of recognizing disease antigens or foreign antigens.

12. The method of claim 11, wherein the thymocytes with T cell receptors which are capable of recognizing disease antigens or foreign antigens are used to treat or prevent cancers, infections, or effects of biological warfare agents.

13. The method of claim 9, wherein the polypeptide of interest causes negative selection of thymocytes having a T cell receptor specificity.

14. The method of claim 13, wherein the negative selection of a T cell receptor specificity reduces or eliminates thymocytes having T cell receptors which are capable of recognizing a self antigen.

15. The method of claim 14, wherein the thymocytes having T cell receptors which are capable of recognizing a self antigen are used to treat or prevent autoimmune disease.

16. A method for eliciting an immune response against disease antigens or foreign antigens by thymic vaccination in a host, comprising:
a) contacting thymic cells with a polypeptide of interest that causes selection of thymocytes having T cell receptor specificity in the thymus capable of recognizing disease antigens or foreign antigens;
b) inducing the thymic cells selected for in (a) to leave the thymus; and
c) expanding the population of disease recognizing thymic cells to a number sufficient to elicit an immune response against the disease antigens or foreign antigens.

17. The method of claim 16, wherein the host is a human or animal.

18. The method of claim 17, wherein the host is a human infant.

19. The method of claim 16, wherein the disease a ntigens or foreign antigens are bacterial, viral, fungal, tumor-associated antigens, oncogene products, parasite antigens and allergens.

20. Expanded thymic cell population produced as in claim 16.

21. A method for preventing or treating autoimmune disease in a host by thymic selection, comprising contacting thymic cells with a polypeptide of interest that reduces or eliminates cells bearing T cell receptors capable of recognizing a self antigen thereby preventing said T cell receptors from leaving the thymus and mediating autoimmune disease.

22. The method of claim 21, wherein the self antigen is capable of mediating autoimmune disease selected from the group consisting of systemic lupus erythematosus, arthritis, thyroidosis, scleroderma, diabetes mellitus, Graves disease and graft versus host disease.

23. A method for eliciting an immune response against disease antigens or foreign antigens by thymic vaccination in a host, comprising:
a) implanting into a host a synthetic thymus comprising stromal elements bearing MHC molecules and polypeptide capable of expanding thymocytes bearing T cell receptors capable of recognizing disease antigens or foreign antigens; and
b) inducing bone marrow progenitor cells to produce a population of thymocytes having T cell receptors capable of eliciting an immune response against disease antigens or foreign antigens.

24. The method of claim 23, wherein the synthetic thymus is implanted subcutaneously or intramuscularly.

25. The method of claim 23, wherein the disease antigens or foreign antigens are bacterial, viral, fungal, tumor-associated antigens, oncogene products, parasite antigens and allergens.

26. A method for preventing or treating autoimmune disease in a host by thymic selection, comprising:
a) implanting into a host a synthetic thymus comprising stromal elements bearing MHC molecules and polypeptide capable of reducing or eliminating T cell receptors on thymic cells that recognize a self antigen; and
b) inducing bone marrow progenitor cells to produce a population of thymocytes having T cell receptors capable of eliciting an immune response to prevent or treat autoimmune disease.

27. The method of claim 26, wherein the self antigen is capable of mediating autoimmune disease selected from the group consisting of systemic lupus erythematosus, arthritis, thyroidosis, sclerodenna, diabetes mellitus, Graves disease and graft versus host disease.

28. The method of claim 27, wherein the synthetic thymus is implanted subcutaneously or intramuscularly.


SEQUENCE LISTING (2 sequences)

SEQ ID NO: 1 - protein, 8 aa, vesicular stomatitis virus
RGYVYQGL

SEQ ID NO: 2 - protein, 15 aa, Artificial Sequence
  peptide linker sequence
GSADDAKKDAAKKDG